(19) United States
(12) Patent Application Publication
(10) Pub. No.: US 2026/0334965 A1
(43) Pub. Date: Oct. 29, 2026

(54) MEDICAL DEVICE

(71) Applicant: ASAHI INTECC CO., LTD., Seto-shi (JP)

(72) Inventors: Makoto NISHIGISHI, Seto-shi (JP); Hirotomo SHIMIZU, Seto-shi (JP)

(73) Assignee: ASAHI INTECC CO., LTD., Seto-shi (JP)

(21) Appl. No.: 19/674,019

(22) Filed: May 11, 2026

Related U.S. Application Data
(63) Continuation of application No. PCT/JP2023/042891, filed on Nov. 30, 2023.

(51) Int. Cl. A61B 17/22; A61B 17/00; A61M 25/00

(52) U.S. Cl. CPC A61B 17/22; A61B 17/00234; A61M 25/0029; A61M 25/0068; A61B 2017/00305; A61B 2017/00778; A61B 2017/22039; A61B 2017/22095

(57) ABSTRACT

The medical device includes a first tube having a first lumen, a second tube placed alongside the first tube and having a second lumen, and a third tube placed alongside the first and second tubes and having a third lumen. The distal end of the second tube is placed closer to the proximal end side than the distal end of the first tube and closer to the proximal end side than the distal end of the third tube, and a first distal end opening for the second lumen to communicate with the outside is formed at the distal end of the second tube, and at the position of the first distal end opening, the first tube and the third tube are separated from each other to form a gap between the first tube and the third tube.

### TECHNICAL FIELD

The disclosed embodiments relate to a medical device.

### BACKGROUND

Medical devices for acquiring ultrasound images are known. For example, patent literatures 1 to 4 describe catheters with an ultrasonic transducer placed at a distal end portion for transmitting and receiving ultrasonic waves toward living tissue. Hereafter, the ultrasonic image acquisition mechanism including ultrasonic transducers will also be referred to as “sensor”. Percutaneous techniques are performed using such medical devices, so that the safety and efficiency of the techniques can be improved.

### CITATION LIST

### Patent Literature

Patent Literature 1: JP 2001-245886 A
Patent Literature 2: JP 2006-20944 A
Patent Literature 3: JP 2016-54797 A
Patent Literature 4: JP 2018-186946 A

### SUMMARY

### Technical Problem

The medical devices described above are configured with multiple lumens by combining multiple tubes or using multi-lumen tubes. However, with medical devices having multiple lumens, depending on the circumferential direction of the medical device within the blood vessel, a situation may arise where a protrusion port of the therapeutic device is not in the direction of the target site of treatment, making the technique difficult. And under these circumstances, there is a problem that changing the circumferential direction of the medical device within the blood vessel (rotating the medical device within the blood vessel) is not always easy, with the risk of false lumen dilation when the medical device is within the false lumen. The techniques described in patent literatures 1 to 4 do not take any of these problems into account.

These problems are not limited to medical devices with built-in sensors, but are also common to medical devices that are configured to allow insertion of sensors and are used in combination with sensors. Moreover, such a problem is also common to medical devices to be inserted into not only blood vascular systems, but also each organ in vivo, such as the lymph gland system, the biliary system, the urinary tract system, the airway system, the digestive organ system, secretory glands, and reproductive organs.

The disclosed embodiments were made to solve at least some of the above-mentioned problems, and are intended to make it easier to direct a therapeutic device in the lumen toward the target site for treatment in a medical device having multiple lumens.

### Solution to Problem

The disclosed embodiments are made in order to solve at least some of the above problems, and can be implemented according to the following aspects.

(1) According to one aspect of the disclosed embodiments, a medical device is provided. The medical device includes a first tube having a first lumen, a second tube placed alongside the first tube and having a second lumen, and a third tube placed alongside the first and second tubes and having a third lumen, wherein the distal end of the second tube is placed closer to the proximal end side than the distal end of the first tube and closer to the proximal end side than the distal end of the third tube, a first distal end opening for the second lumen to communicate with the outside is formed at the distal end of the second tube, and at the position of the first distal end opening, the first tube and the third tube are separated from each other to form a gap between the first tube and the third tube.

### BRIEF DESCRIPTION OF THE DRAWINGS

FIG. 1 is an explanatory view illustrating a configuration of a medical device.

FIG. 2 is an explanatory view illustrating a configuration of a medical device.

FIG. 3 is a transverse sectional view of a catheter along line A-A in FIG. 1.

FIG. 4 is a transverse sectional view of the catheter along line B-B in FIG. 1.

FIG. 5 is a transverse sectional view of the catheter along line C-C in FIG. 1.

FIG. 6 is a transverse sectional view of the catheter along line D-D in FIG. 1.

FIG. 7 is a transverse sectional view of the catheter along line E-E in FIG. 1.

FIG. 8 is a diagram for describing a method for using the catheter.

FIG. 9 is a diagram for describing a method for using the catheter.

FIG. 10 is an enlarged view of a portion of the distal end side of the catheter.

FIG. 11 is a transverse sectional view of the catheter along line F-F in FIG. 10.

FIG. 12 is a transverse sectional view of the catheter along line G-G in FIG. 10.

FIG. 13 shows a therapeutic device directed in the first direction.

FIG. 14 shows the therapeutic device directed in the second direction.

FIG. 15 shows the catheter and combined devices in a first technique.

FIG. 16 shows the catheter and combined devices in a second technique.

FIG. 17 shows the catheter and combined devices in a third technique.

FIG. 18 is an enlarged view of a portion on the distal end side of the catheter of a second embodiment.

FIG. 19 is an enlarged view of a portion on the distal end side of the catheter of a third embodiment.

FIG. 20 is a transverse sectional view of the catheter along line H-H in FIG. 19.

FIG. 21 is a transverse sectional view of a catheter of a fourth embodiment.

FIG. 22 is an enlarged view of a portion on the distal end side of a catheter of a fifth embodiment.

### DETAILED DESCSRIPTION

### First Embodiment

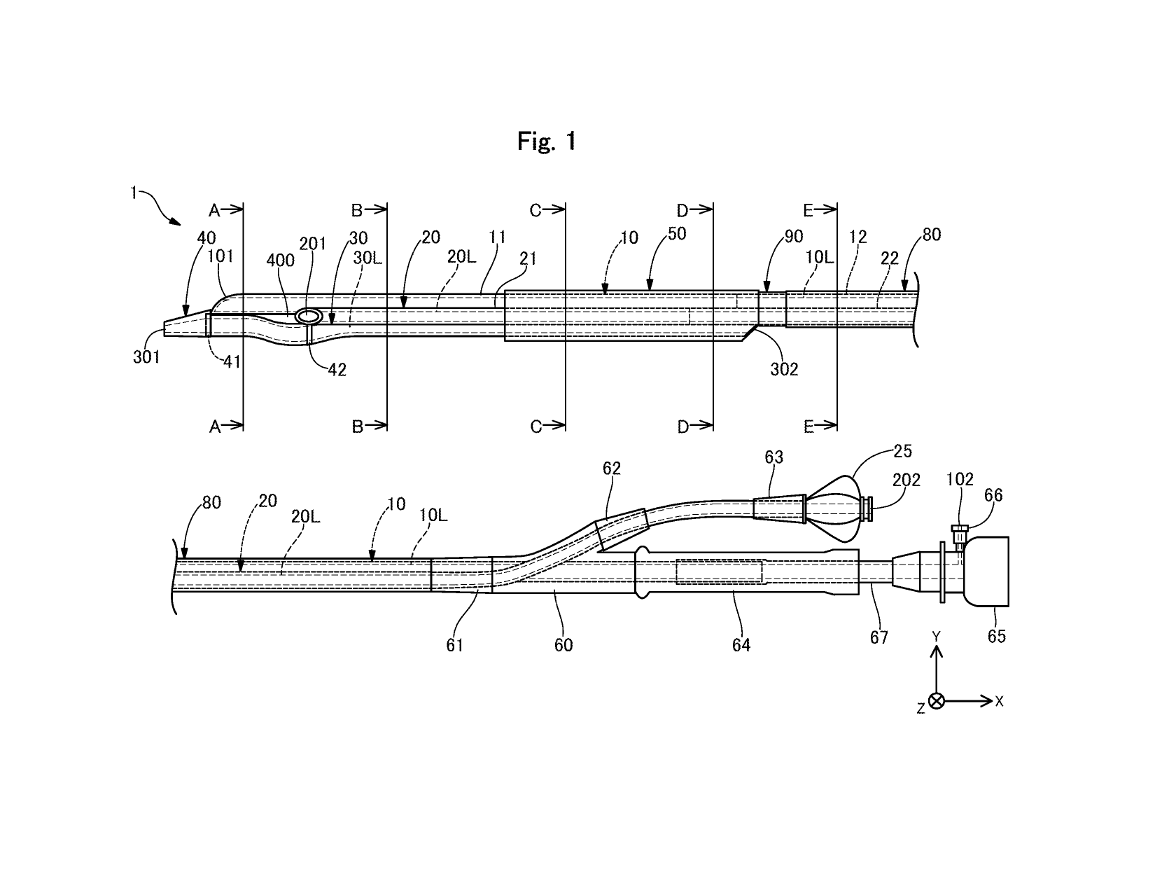

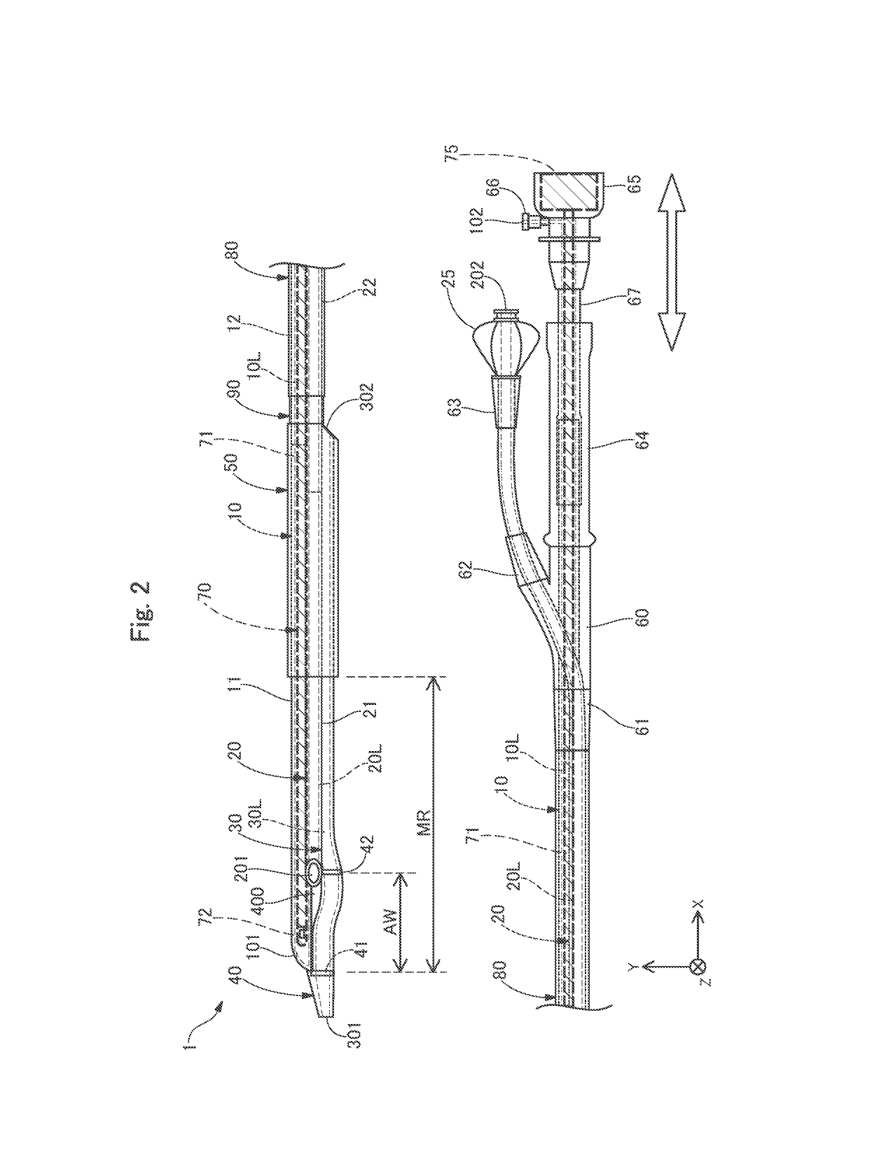

FIGS. 1 and 2 are explanatory views illustrating the configuration of a medical device 1. The medical device 1 of this embodiment is a catheter used to treat a lesion in a living body lumen, such as a CTO (Chronic Total Occlusion) in a blood vessel, for example. Henceforth, the medical device 1 is also referred to as “catheter 1”. As shown in FIGS. 1 and 2, the catheter 1 includes a sensor tube 10, an OTW (Over The Wire) tube 20, an RX (Rapid Exchange) tube 30, a distal tip 40, a first marker 41, a second marker 42, a first outer tube 50, a branch connector 60, a first reinforcement member 61 to a third reinforcement member 63, an outer tubular member 64, an inner tubular member 67, a connector 65, a connector 25, a sensor 70, a second outer tube 80, and a heat shrink tube 90.

In FIG. 1, the illustration of the sensor 70 is omitted to describe the configurations of the tubes and the lumens in the tubes. In FIG. 2, the sensor 70 built into a sensor lumen 10L in the sensor tube 10 is represented by a dashed line with shaded hatching.

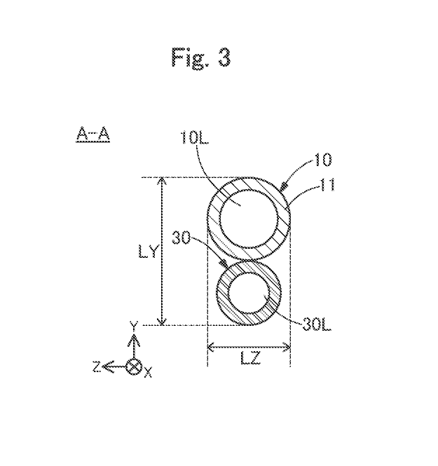

FIGS. 1 and 2 include portions in which the relative ratio of the size of each component differs from that of the actual size, for convenience of description. FIGS. 1 and 2 also include portions in which a part of each component is exaggerated. Further, FIGS. 1 and 2 illustrates XYZ axes that are orthogonal to each other. The X-axis corresponds to the longitudinal direction of the catheter 1, the Y-axis corresponds to the height direction of the catheter 1, and the Z-axis corresponds to the width direction of the catheter 1. The left side (-X-axis direction) of FIGS. 1 and 2 and FIG. 2 is referred to as the “distal end side” of the catheter 1 and each component, while the right side (+X-axis direction) of FIGS. 1 and 2 is referred to as the “proximal end side” of the catheter 1 and each component. Moreover, of both ends in the longitudinal direction (X-axis direction) of the catheter 1 and each component, one end positioned on the distal end side is referred to as a “distal end”, and the other end positioned on the proximal end side is referred to as a “proximal end”. Further, the distal end and the vicinity thereof are referred to as a “distal end portion”, and the proximal end and the vicinity thereof are referred to as a “proximal end portion”. The portion on the distal end side is inserted into a living body, and the portion on the proximal end side is operated by a surgeon such as a doctor. These points are also common to FIG. 3 and the Figs following FIG. 3.

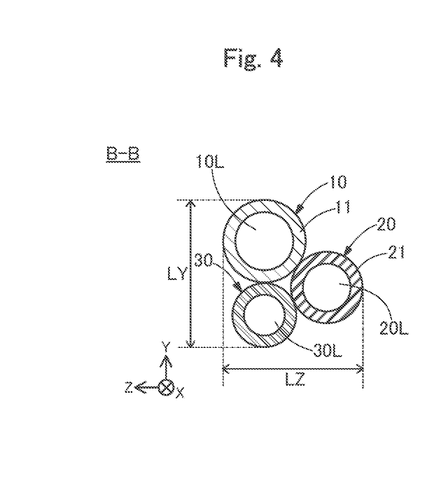

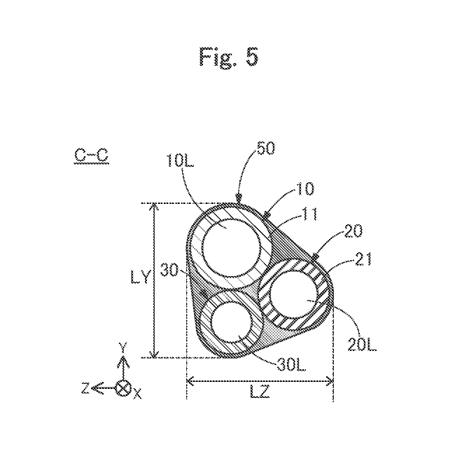

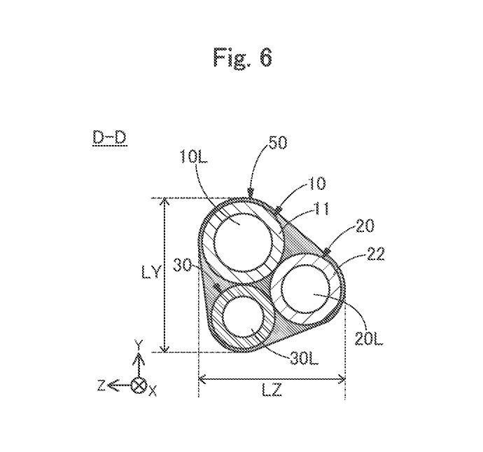

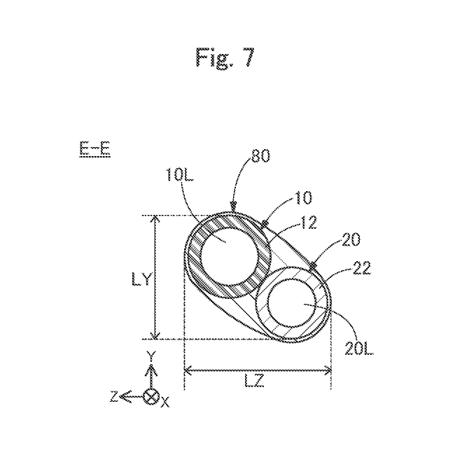

FIGS. 3-7 show the transverse sections of the catheter 1. FIG. 3 is a transverse sectional view of the catheter 1 along line A-A in FIG. 1. FIG. 4 is a transverse sectional view of the catheter 1 along line B-B in FIG. 1. FIG. 5 is a transverse sectional view of the catheter 1 along line C-C in FIG. 1. FIG. 6 is a transverse sectional view of the catheter 1 along line D-D in FIG. 1. FIG. 7 is a transverse sectional view of the catheter 1 along line E-E in FIG. 1. The following describes the configuration of the catheter 1 with reference to FIGS. 1-7.

The sensor tube 10 is a cylindrical member (tubular body) with a long external shape. The sensor tube 10 extends in a straight line along the longitudinal direction (X-axis direction) of the catheter 1, parallel to the OTW tube 20 and the RX tube 30. Inside the sensor tube 10, a sensor lumen 10L (dashed line) to accommodate the sensor 70 is formed. The sensor lumen 10L is a lumen for the sensor 70.

The distal end of the sensor tube 10 is located at the same or slightly proximal end side as the distal end of the RX tube 30 in the longitudinal direction of the catheter 1. At the distal end of the sensor tube 10, a distal end opening 101 for the distal end of the sensor lumen 10L to communicate with the outside is formed. The distal end opening 101 is a fluid outlet for wetting the inside of the sensory lumen 10L. The proximal end of the sensor tube 10 is located closer to the proximal end side than the proximal end of the OTW lumen 20L and the proximal end of the RX tube 30 in the longitudinal direction of the catheter 1. The proximal end portion of the sensor tube 10 is inserted into the lumen of an inner tubular member 67 and is held movable relative to the inner tubular member 67. In other words, the inner tubular member 67 is placed along the outer peripheral surface of the proximal end portion of the sensor tube 10 and is movable in the longitudinal direction of the sensor tube 10. The lumen of the inner tubular member 67 communicates with the sensor lumen 10L. The proximal end portion of the inner tubular member 67 is fixed to a connector 65. A fluid supply part 66 is attached to the connector 65, and the fluid supply part 66 has a proximal end opening 102 formed for the lumen of the inner tubular member 67 and the proximal end of the sensor lumen 10L to communicate with the outside. The proximal end opening 102 is a fluid supply port to the lumen of the inner tubular member 67 and the sensor lumen 10L.

As shown in FIG. 1, the sensor tube 10 has a distal end side tube 11 placed on the distal end side and a proximal end side tube 12 placed closer to the proximal end side than the distal end side tube 11. Both the distal end side tube 11 and the proximal end side tube 12 are cylindrical members (tubular bodies) with a long external shape. The distal end side tube 11 and the proximal end side tube 12 are connected inside the first outer tube 50 in the longitudinal direction. That is, the distal end side tube 11 and the proximal end side tube 12 each constitute a part of the sensory lumen 10L.

The OTW tube 20 is a cylindrical member (tubular body) having a long external shape. The OTW tube 20 extends in a straight line along the longitudinal direction of the catheter 1, parallel to the sensor tube 10 and the RX tube 30, on the distal end side of the branch connector 60. Inside the OTW tube 20, an OTW lumen 20L (dashed line) to accommodate a therapeutic device (e.g., plasma guidewire or penetrating guidewire) is formed. The OTW lumen 20L is a so-called over-the-wire (OTW) type lumen that has no opening at a portion that is placed in a living body lumen when the catheter 1 is used.

The distal end of the OTW tube 20 is located closer to the proximal end side than the distal end of the sensor tube 10, and closer to the proximal end side than the distal end of the RX tube 30 in the longitudinal direction of the catheter 1. At the distal end of the OTW tube 20, a distal end opening 201 for the distal end of the OTW lumen 20L to communicate with the outside is formed. The distal end opening 201 is a device protrusion port for the therapeutic device to protrude into the living tissue. The distal end opening 201 is elliptical in shape with the distal end portion of the OTW tube 20 cut diagonally. The “diagonal” is the direction that intersects the longitudinal direction of the catheter 1, and is the direction toward a gap 400 from the proximal end side to the distal end side. The distal end opening 201 is inclined in the direction toward the gap 400 from the proximal end side to the distal end side. This makes it easier for the therapeutic device to reach the living tissue surrounding the catheter 1 when the catheter 1 is in use. The proximal end of the OTW tube 20 is located closer to the distal end side than the proximal end of the sensor tube 10 and closer to the proximal end side than the proximal end of the RX tube 30 in the longitudinal direction of the catheter 1. On the proximal end side of the OTW tube 20, the first reinforcement member 61, the branch connector 60, the second reinforcement member 62, the third reinforcement member 63, and the connector 25 are mounted from the distal end side to the proximal end side. Details are described below. The connector 25 has a proximal end opening 202 formed for the proximal end of the OTW lumen 20L to communicate with the outside. The proximal end opening 202 is a device insertion port for inserting a therapeutic device into the OTW lumen 20L.

As shown in FIG. 1, the OTW tube 20 has a distal end side tube 21 placed on the distal end side and a proximal end side tube 22 placed closer to the proximal end side than the distal end side tube 21. Both the distal end side tube 21 and the proximal end side tube 22 are cylindrical members (tubular bodies) with a long external shape. The distal end side tube 21 and the proximal end side tube 22 are connected inside the first outer tube 50 in the longitudinal direction. That is, the distal end side tube 21 and the proximal end side tube 22 each constitute a part of the OTW lumen 20L.

The RX tube 30 is a cylindrical member (tubular body) with a long external shape. The RX tube 30 extends in a straight line along the longitudinal direction of the catheter 1, parallel to the sensor tube 10 and the OTW tube 20. Inside the RX tube 30, an RX lumen 30L (dashed line) to accommodate a workhorse wire is formed. The RX lumen 30L includes the lumen of the RX tube 30 and the lumen of the distal tip 40. Therefore, the distal end of the RX lumen 30L is located closer to the distal end side than the distal end of the sensor lumen 10L.

The distal end of the RX tube 30 is located at the same or slightly on the distal end side as the distal end of the sensor tube 10 in the longitudinal direction of the catheter 1. A hollow distal tip 40 is joined to the distal end portion of the RX tube 30. At the distal end of the distal tip 40, a distal end opening 301 for the distal end of the RX lumen 30L to communicate with the outside is formed. The distal end opening 301 is a wire insertion port for inserting a workhorse wire into the RX lumen 30L. The proximal end of the RX tube 30 is located closer to the distal end side than the proximal end of the sensor tube 10 and the proximal end of the OTW tube 20 in the longitudinal direction of the catheter 1. At the proximal end of the RX tube 30, a proximal end opening 302 for the proximal end of the RX lumen 30L to communicate with the outside is formed. The proximal end opening 302 is a wire pullout port for pulling a workhorse wire outward. The proximal end opening 302 is elliptical in shape with the proximal end of the RX tube 30 cut diagonally and oriented crosswise to the longitudinal direction of the catheter 1. This makes it easier to pull the workhorse wire out of the proximal end opening 302 when the catheter 1 is in use.

The distal tip 40 is a tubular member having radiopacity and an outer diameter that expands from the distal end side to the proximal end side. The distal tip 40 is located at the distal end of the catheter 1 by being joined to the distal end portion of the RX tube 30 and travels through the living body lumen ahead of the other members. The lumen of the distal tip 40 communicates with the RX lumen 30L of the RX tube 30, and a distal end opening 301 for the distal end of the RX lumen 30L to communicate with the outside is formed at the distal end of the distal tip 40, as described above.

The first marker 41 and the second marker 42 are ring-shaped members having radiopacity. The first marker 41 is placed so that the proximal end of the first marker 41 and the proximal end of the distal tip 40 are at the same position in the longitudinal direction of the catheter 1. The first marker 41 is buried between the outer peripheral surface of the RX tube 30 and the inner peripheral surface of the distal tip 40. The second marker 42 is placed so that the proximal end of the second marker 42 and a central portion of the distal end opening 201are at the same position in the longitudinal direction of the catheter 1. The second marker 42 is joined to the outer peripheral surface of the RX tube 30. For joining of the first marker 41 and the second marker 42, resin-to-resin bonding by thermal melting or bonding with an adhesive such as an epoxy adhesive can be employed. The second marker 42 is visible to the naked eye. Placing the first marker 41 and the second marker 42 on the RX tube 30 instead of the sensor tube 10 can prevent sensing (acquisition of image information) by a sensor 70 from being hindered by the first marker 41 and the second marker 42.

As shown in FIG. 3, in a transverse section along line A-A, the sensor tube 10 (specifically, the distal end side tube 11) and the RX tube 30 are placed. In transverse section A-A, the sensor tube 10 and the RX tube 30 are joined with a portion of the outer peripheral surface of the sensor tube 10 and a portion of the outer peripheral surface of the RX tube 30 in contact with each other. As shown in FIG. 4, in a transverse section along line B-B, the sensor tube 10 (specifically the distal end side tube 11), the OTW tube 20 (specifically the distal end side tube 21), and the RX tube 30 are placed. In transverse section B-B, the three tubes 10, 20, 30 are joined together with a portion of the outer peripheral surface of the sensor tube 10, a portion of the outer peripheral surface of the OTW tube 20, and a portion of the outer peripheral surface of the RX tube 30 in contact with each other. In a predetermined area located between transverse section A-A and transverse section B-B, a gap 400 is formed between the sensor tube 10 and the RX tube 30. The gap 400 is discussed below.

As shown in FIG. 5, in a transverse section along line C-C, the sensor tube 10 (specifically the distal end side tube 11), the OTW tube 20 (specifically the distal end side tube 21), and the RX tube 30 are covered by the first outer tube 50. Specifically, each outer peripheral surface of the three tubes 10, 20, 30 is covered by the melt-molded first outer tube 50, thereby integrally fixing the three tubes 10, 20, 30. As shown in FIG. 6, in a transverse section along line D-D, the sensor tube 10 (specifically the distal end side tube 11), the OTW tube 20 (specifically the proximal end side tube 22), and the RX tube 30 are covered by the first outer tube 50 similarly to FIG. 5. As shown in FIG. 7, in a transverse section along line E-E, the sensor tube 10 (specifically the proximal end side tube 12) and the OTW tube 20 (specifically the proximal end side tube 22) are covered by a second outer tube 80. Specifically, each outer peripheral surface of the two tubes 10, 20 is covered with the melt-molded second outer tube 80, thereby integrally fixing the two tubes 10, 20.

Joining of the sensor tube 10, the OTW tube 20, and the RX tube 30 in transverse section A-A and transverse section B-B may be done using any adhesive, such as an epoxy adhesive, or may be heat welded. In transverse section A-A, transverse section B-B, transverse section C-C, and transverse section D-D, the height LY of the catheter 1 is greater than the width LZ of the catheter 1. On the other hand, in transverse section E-E, the height LY of the catheter 1 is smaller than the width LZ of the catheter 1. As shown in FIGS. 3-7, the size relationship of the outer diameters of the three tubes 10, 20, 30 is as follows: outer diameter of sensor tube 10 > outer diameter of OTW tube 20 > outer diameter of RX tube 30. The size relationship of the inner diameters (lumens) of the three tubes 10, 20, 30 is as follows: inner diameter of sensor lumen 10L > inner diameter of OTW lumen 20L > inner diameter of RX lumen 30L. However, these size relationships of the outer and inner diameters are only examples and may be changed as desired.

The outer shape of the catheter 1 in transverse section A-A and transverse section B-B follows the contours of the two tubes 10, 30 (or three tubes 10, 20, 30) placed next to each other, with a neck (concave portion) formed at the adjacent portion of each tube. Transverse section C-C and transverse section D-D, in other words, the outer shape of the catheter 1 at the portion covered by the first outer tube 50 is triangular with rounded corners (rounded triangle). Transverse section E-E, in other words, the outer shape of the catheter 1 at the portion covered by the second outer tube 80, is elliptical in shape.

Returning to FIG. 1, description is continued. The three tubes 10, 20, 30 (specifically, sensor tube 10, OTW tube 20, and RX tube 30) are fixed by three tubes 90, 50, 80.

A heat shrink tube 90 is placed between the first outer tube 50 and the second outer tube 80 in the longitudinal direction of the catheter 1. The heat shrink tube 90 covers the sensor tube 10 (specifically, a portion on the distal end side of the proximal end side tube 12) and the OTW tube 20 (specifically, a portion on the distal end side of the proximal end side tube 22), bundling the two tubes 10, 20. The heat shrink tube 90 does not cover the RX tube 30. The RX tube 30 is placed along the outer peripheral surface of the heat shrink tube 90, with the outer peripheral surface of the heat shrink tube 90 and the outer peripheral surface of the RX tube 30 in contact with each other. The distal end of the heat shrink tube 90 is located closer to the proximal end side than the distal end of the first outer tube 50 and closer to the distal end side than the proximal end opening 302. Specifically, the distal end portion of the heat shrink tube 90 is covered by the first outer tube 50. The proximal end of the heat shrink tube 90 is located closer to the proximal end side than the distal end of the second outer tube 80 and closer to the distal end side than the first reinforcement member 61. Specifically, the proximal end portion of the heat shrink tube 90 is covered by the second outer tube 80. In other words, the middle portion of the heat shrink tube 90 in the longitudinal direction of the catheter 1 is not covered by the first outer tube 50 or the second outer tube 80.

The first outer tube 50 is placed closer to the distal end side than the heat shrink tube 90 in the longitudinal direction of the catheter 1. The first outer tube 50 is provided at the section being closer to the proximal end side than the distal end opening 201 and including the three tubes 10, 20, 30 that extend side by side. In the example in FIG. 1, the distal end of the first outer tube 50 is located near the center between the distal end opening 201 and the proximal end opening 302. The proximal end of the first outer tube 50 is located near the proximal end of the proximal end opening 302. Thus, the first outer tube 50 is preferably provided at a position away from the distal end opening 201 toward the proximal end side. This way can prevent sensing (acquisition of image information) by the sensor 70 inserted in the sensor lumen 10L from being hindered by the first outer tube 50. The first outer tube 50 covers the distal end portion of the heat shrink tube 90, the sensor tube 10 exposed from the distal end of the heat shrink tube 90 (specifically, a portion on the proximal end side of the distal end side tube 11), the OTW tube 20 exposed from the distal end of the heat shrink tube 90 (specifically, a portion on the proximal end side of the distal end side tube 21, and a portion on the distal end side of the proximal end side tube 22), and the RX tube 30, and fix them together. As shown in FIGS. 5 and 6, the first outer tube 50 has a rounded triangular outer shape and has thick-walled sections melt-molded along the outer peripheral surface of the three tubes 10, 20, 30.

The second outer tube 80 is placed closer to the proximal end side than the heat shrink tube 90 in the longitudinal direction of the catheter 1. The second outer tube 80 is provided at the section being closer to the proximal end side than the proximal end opening 302 and including the two tubes 10, 20 that extend side by side. In the example in FIG. 1, the distal end of the second outer tube 80 is located at a portion slightly away from the proximal end opening 302 toward the proximal end side. The proximal end of the second outer tube 80 is located inside the first reinforcement member 61. The second outer tube 80 covers the proximal end portion of the heat shrink tube 90, the sensor tube 10 exposed from the proximal end of the heat shrink tube 90 (specifically, a portion on the proximal end side of the proximal end side tube 12), and the OTW tube 20 exposed from the proximal end of the heat shrink tube 90 (specifically, a portion of the proximal end side tube 22), and fixes them together. As shown in FIG. 7, the second outer tube 80 has an elliptical outer shape and has a thick-walled section melt-molded along the outer peripheral surface of the two tubes 10, 20.

The proximal end portion of the catheter 1 has a first reinforcement member 61 and a branch connector 60 provided from the distal end side to the proximal end side. The first reinforcement member 61 is a cylindrical member placed closer to the distal end side than the branch connector 60. The first reinforcement member 61 reinforces the distal end side of the branch connector 60 by covering the outer periphery of the second outer tube 80 that bundles the sensor tube 10 and the OTW tube 20. The branch connector 60 is mounted closer to the proximal end side than the first reinforcement member 61. The branch connector 60 is a member with a bifurcated lumen. The OTW tube 20 is inserted into one lumen of the branch connector 60. The sensor tube 10 is inserted into the other lumen of the branch connector 60.

On one side of the branch connector 60, a second reinforcement member 62, a third reinforcement member 63, and a connector 25 are provided from the distal end side to the proximal end side. The second reinforcement member 62 is a cylindrical member placed closer to the proximal end side than the branch connector 60. The second reinforcement member 62 reinforces the proximal end side of the branch connector 60 by covering the outer periphery of the OTW tube 20 inserted into the branch connector 60. The third reinforcement member 63 is a cylindrical member placed closer to the distal end side than the connector 25. The third reinforcement member 63 reinforces the distal end side of the connector 25 by covering the outer periphery of the OTW tube 20 inserted into the connector 25. The connector 25 is a member joined to the proximal end portion of the OTW tube 20. The connector 25 has a pair of blades for the surgeon to grasp. At the proximal end of the connector 25, a proximal end opening 202 (device insertion port) for the proximal end of the OTW lumen 20L to communicate with the outside is formed.

On the other side of the branch connector 60, an outer tubular member 64, an inner tubular member 67, and a connector 65 are provided from the distal end side to the proximal end side. The outer tubular member 64 is a cylindrical member placed closer to the proximal end side than the branch connector 60. The inner tubular member 67 is inserted into the lumen of the outer tubular member 64 and is held movable relative to the outer tubular member 64. Protrusions are provided on the inner peripheral surface of the proximal end portion of the outer tubular member 64 and on the outer peripheral surface of the distal end portion of the inner tubular member 67, respectively. The engagement of these protrusions prevents the inner tubular member 67 from detaching from the outer tubular member 64 as it moves relative to the outer cylindrical member 64. The proximal end portion of the sensor tube 10 is inserted into the lumen of the outer tubular member 64. The sensor tube 10 is held movable relative to the inner tubular member 67 (in other words, the inner tubular member 67 is placed along the outer peripheral surface of the proximal end portion of the sensor tube 10 and movable in the longitudinal direction of the sensor tube 10). The connector 65 is a member joined to the proximal end portion of the inner tubular member 67. On the proximal end side of the connector 65, an enclosure to accommodate a connection terminal 75 of the sensor 70 is provided. A fluid supply part 66 is provided on the outer peripheral surface of the connector 65, wherein the proximal end opening 102 for the proximal end of the sensor lumen 10L and the proximal end of the lumen of the inner tubular member 67 to communicate with the outside is formed. The lumen of the inner tubular member 67 is a portion of the sensor lumen 10L.

The sensor 70 (FIG. 2) is an imaging sensor for acquiring image information. As shown in FIG. 2, the sensor 70 includes a main body part 71, the probe 72, and the connection terminal 75. The main body part 71 is a long member extending along the longitudinal direction of the catheter 1. A driving cable (coaxial wire) that electrically connects the probe 72 to the connection terminal 75 is built inside the main body part 71. The probe 72 includes an ultrasonic probe (also referred to as an ultrasonic vibrator, a piezoelectric body, an ultrasonic transmitting/receiving element, or an ultrasonic element) that emits ultrasonic waves to a living tissue and receives ultrasonic waves reflected during propagation in the living tissue. The probe 72 is also referred to as an imaging core or transducer. The connection terminal 75 is a terminal that electrically connects the sensor 70 to an externally provided console terminal. The connection terminal 75 is provided at the proximal end of the main body part 71 and is housed in the enclosure of the connector 65.

The sensor 70 is electrically connected to an external console terminal via the connection terminal 75, receives power supply from the console terminal, and outputs detection signals obtained by the use of a probe 72 to the console terminal. This allows the console terminal to display image information based on the detection signals obtained by the use of the probe 72. As shown in FIG. 2, the sensor 70 is fixed to the connector 65. As indicated by the white arrows in FIG. 2, a surgeon can move the position of the probe 72 of the sensor 70 within a range MR from the distal end of the sensor lumen 10L to the distal end of the first outer tube 50, in other words, within the predetermined range MR including the distal end opening 201 by grasping the connector 65 and sliding it back and forth (in the direction of the white arrow). Henceforth, the range MR is also referred to as “movable range MR”. A portion of the catheter 1, which is particularly suitable for sensing (acquiring image information) by the sensor 70, is also referred to as “acoustic window AW”. As shown in FIG. 2, the acoustic window AW is a section of catheter 1 between the first marker 41 and the second marker 42.

A distal end side tube 11 of the sensor tube 10, a distal end side tube 21 of the OTW tube 20, and the RX tube 30 can be formed of a thermoplastic resin such as polyethylene resin, polypropylene resin, and polyurethane, and a flexible material such as polyvinyl chloride, ethylene-vinyl acetate copolymer, cross-linked ethylene-vinyl acetate copolymer, polyamide elastomer, polyolefin elastomer, polyurethane elastomer, silicone rubber, and latex rubber. The distal end side tube 11 of the sensor tube 10, the distal end side tube 21 of the OTW tube 20, and the RX tube 30 may be formed of the same material or of different materials.

The proximal end side tube 12 of the sensor tube 10 and the proximal end side tube 22 of the OTW tube 20 can be formed of a resin having high stiffness, such as nylon resin, polyester resin, and PEEK resin. The melting points of the proximal end side tube 12 of the sensor tube 10 and the proximal end side tube 22 of the OTW tube 20 are higher than those of the tubes 11, 21, 30 described above. The proximal end side tube 12 of the sensor tube 10 and the proximal end side tube 22 of the OTW tube 20 may be formed of the same material or of different materials.

In the catheter 1 of this embodiment, a section is provided where a portion on the proximal end side of the flexible RX tube 30 is overlapped with the highly stiff proximal end side tube 12 and the proximal end side tube 22 (FIG. 1), thereby achieving a gradual change in the stiffness of the catheter 1. The gradual change in the stiffness of the catheter 1 can be paraphrased as reducing the stiffness gap of the catheter 1. This can prevent kinking of the catheter 1. Any one or more of the distal end side tube 11 and the proximal end side tube 12 of the sensor tube 10, the distal end side tube 21 and the proximal end side tube 22 of the OTW tube 20, and the RX tube 30 may have a multi-layer configuration with overlapping tubes made of different materials.

The distal tip 40, the first marker 41, and the second marker 42 can be formed of radiopaque resin materials or metal materials. In the case of using a radiopaque resin material, for example, a radiopaque material such as bismuth trioxide, tungsten, or barium sulfate is mixed with a polyamide resin, a polyolefin resin, a polyester resin, a polyurethane resin, a silicone resin, a fluororesin, or the like, so that they can be formed. In the case of using a radiopaque metal material, for example, gold, platinum, tungsten, an alloy containing these elements (for example, a platinum-nickel alloy) or the like can be used, so that they can be formed. The distal tip 40, the first marker 41, and the second marker 42 may be formed of the same material or different materials. If the distal tip 40, the first marker 41, and the second marker 42 are made of metal, the images of the distal tip 40, the first marker 41, and the second marker 42 can be obtained not only in angiographic images, but also in the image acquired by the sensor 70.

The branch connector 60, the first reinforcement member 61 to the third reinforcement member 63, the outer tubular member 64, the inner tubular member 67, the connector 65, and the connector 25 can be formed of known resin materials. The branch connector 60, the first reinforcement member 61 to the third reinforcement member 63, the outer tubular member 64, the inner tubular member 67, the connector 65, and the connector 25 may be formed of the same material or of different materials.

The heat shrink tube 90 is formed of a thermoplastic nylon elastomer resin (e.g., polyamide elastomer). The heat shrink tube 90 has the property of not melting but shrinking when heated to a predetermined temperature range. In addition, the heat shrink tube 90 has improved adhesion (the property of being more easily attached to other substances) when heated compared to when not heated. The heat shrink tube 90 may be formed of polyolefin, FEP (Fluorinated Ethylene Propylene), or silicone.

The first outer tube 50 and the second outer tube 80 are formed of a thermoplastic nylon elastomer resin. Unlike the heat shrink tube 90, the first outer tube 50 and the second outer tube 80 have the property of melting when heated. In the example of this embodiment, the first outer tube 50 is made of a resin with a lower Shore hardness than the second outer tube 80. However, the first outer tube 50 and the second outer tube 80 may be formed of the same material or of different materials.

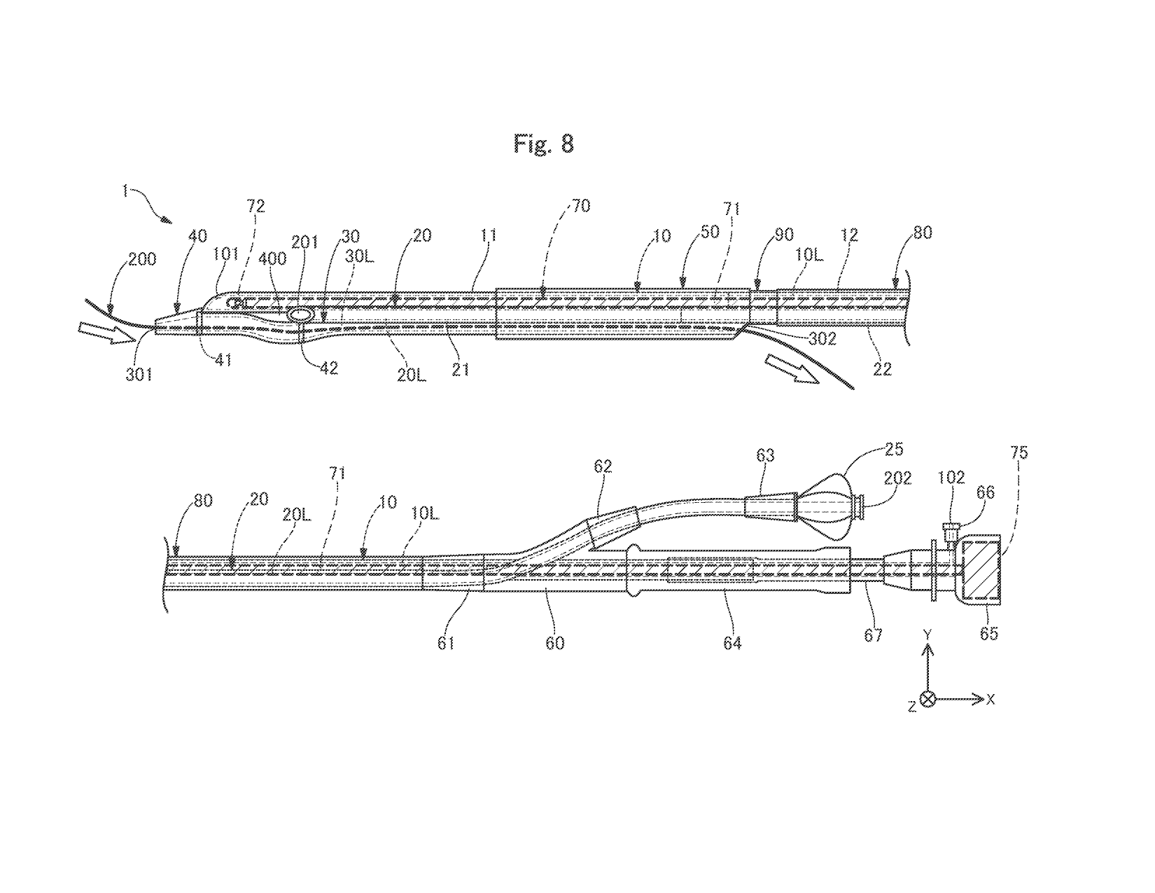

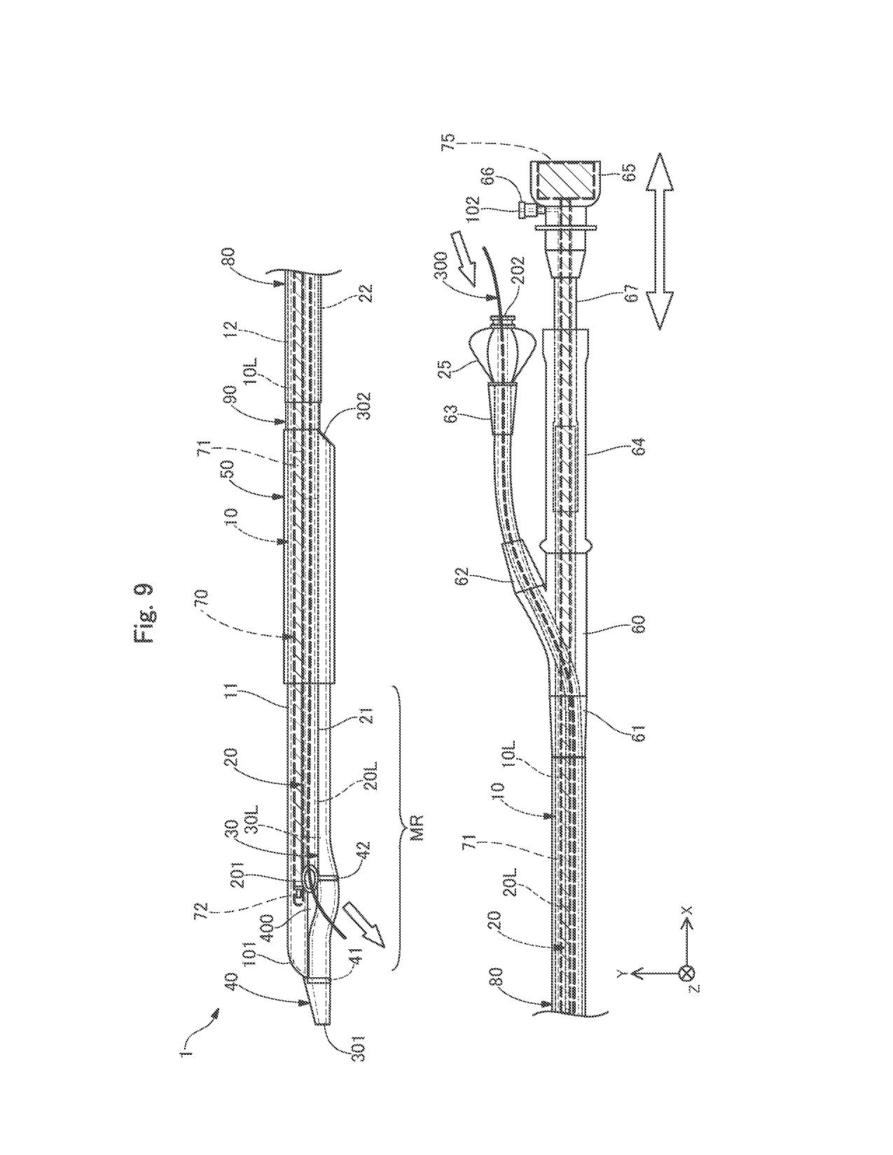

FIGS. 8 and 9 are diagrams for describing a method for using the catheter 1. Procedures a1 through a6, shown below, illustrate a case of recanalizing a CTO (lesion) in a blood vessel by a forward approach. However, the catheter 1 may also be used in a reverse approach and may also be used for techniques other than CTO recanalization.
(a1) A surgeon inserts a workhorse wire 200 into the blood vessel and delivers the distal end portion of the workhorse wire 200 close to CTO.
(a2) The surgeon inserts the proximal end portion of the workhorse wire 200 from the distal end opening 301 of the catheter 1, passes it through the RX lumen 30L, and pulls it out from the proximal end opening 302 of the catheter 1 (FIG. 8).
(a3) The surgeon pushes the catheter 1 along the workhorse wire 200 into the blood vessel and delivers the distal end portion of the catheter 1 close to CTO. In Procedure a3, the catheter 1 may be delivered close to CTO by passing it through a guiding catheter that has been inserted into the blood vessel beforehand along the workhorse wire 200.
(a4) The surgeon grasps and slides the connector 65 back and forth (FIG. 9: in the direction of the white arrow) to check the image displayed on the console terminal while adjusting the position of the probe 72 of the sensor 70 within the movable range MR, thereby adjusting the positions of the CTO and the distal end opening 201 while checking the orientation of the CTO and the distal end opening 201. Here, the position means a position in the direction of blood vessel extension. The orientation means an orientation in the circumferential direction of the inner wall of a blood vessel.
(a5) The surgeon inserts the distal end portion of a therapeutic device 300 from the proximal end opening 202 of the catheter 1 to pass it through the OTW lumen 20L, and protrudes it from the distal end opening 201 of the catheter 1 (FIG. 9).
(a6) The surgeon treats the CTO using the therapeutic device 300 while adjusting the position of the probe 72 of the sensor 70 within the movable range MR as necessary and checking images displayed on the console terminal. As mentioned above, any device such as a plasma guidewire or a penetrating guidewire can be used as the therapeutic device 300.

The sensor tube 10, the OTW tube 20, and the RX tube 30 are also collectively referred to as “shafts”. The sensor tube 10 corresponds to the “first tube” and the sensor lumen 10L corresponds to the “first lumen”. The OTW tube 20 corresponds to the “second tube” and the OTW lumen 20L corresponds to the “second lumen”. The RX tube 30 corresponds to the “third tube” and the RX lumen 30L corresponds to the “third lumen”. The distal end opening 201 of the OTW lumen 20L corresponds to the “first distal end opening”. The distal end opening 301 of the RX lumen 30L corresponds to the “second distal end opening”. The “same” and “equal” in this embodiment are not limited to cases of strict agreement, but are meant to allow for differences due to manufacturing errors and the like. As used herein, “constant” is synonymous with “generally constant”, and means to be generally constant while allowing fluctuations due to manufacturing errors and the like.

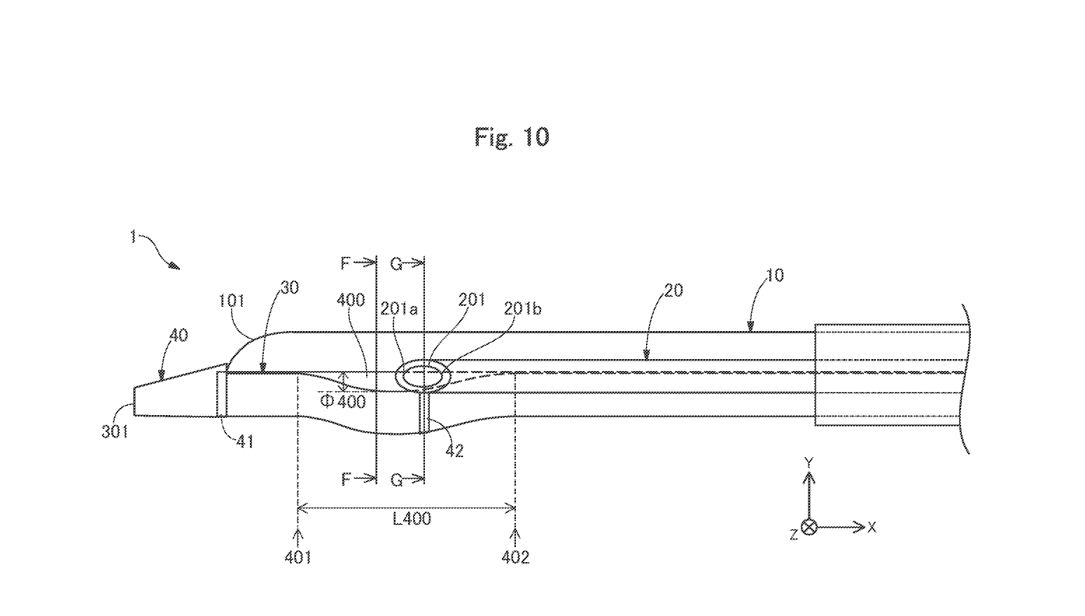

FIG. 10 is an enlarged view of a portion on the distal end side of the catheter 1. In FIG. 10, the illustration of the sensor lumen 10L, the OTW lumen 20L, and the RX lumen 30L is omitted. In FIG. 10, portions of the sensor tube 10 and the RX tube 30, located behind the OTW tube 20, are illustrated with dashed lines.

At the distal end portion of the catheter 1, a gap 400 is formed between the sensor tube 10 (first tube) and the RX tube 30 (third tube). The gap 400 is a void formed between the outer peripheral surface of the sensor tube 10 and the outer peripheral surface of the RX tube 30, since these outer peripheral surfaces are separated from each other. In the example in FIG. 10, the sensor tube 10 is moved straight along the longitudinal direction of the catheter 1, while a portion of the RX tube 30 is curved in the direction away from the sensor tube 10 (-Y axis direction) so as to be separated from the sensor tube 10, forming the gap 400.

As shown in FIG. 10, the gap 400 is formed at the position of the distal end opening 201 (first distal end opening) for the OTW lumen 20L to communicate with the outside. Here, the position of the distal end opening 201 means any area that includes the distal end opening 201. In the longitudinal direction of the catheter 1, the distal end of the gap 400 is referred to as “gap distal end 401” and the proximal end of the gap 400 is referred to as “gap proximal end 402”. In the example in FIG. 10, the gap distal end 401 is located away from the distal end of the sensor tube 10 toward the proximal end side and located closer to the distal end side than the distal end 201a of the distal end opening 201. The gap proximal end 402 is located closer to the proximal end side than the proximal end 201b of the distal end opening 201. The RX tube 30 and the sensor tube 10 are separated from each other, so that the gap 400 is formed between the gap distal end 401 and the gap proximal end 402.

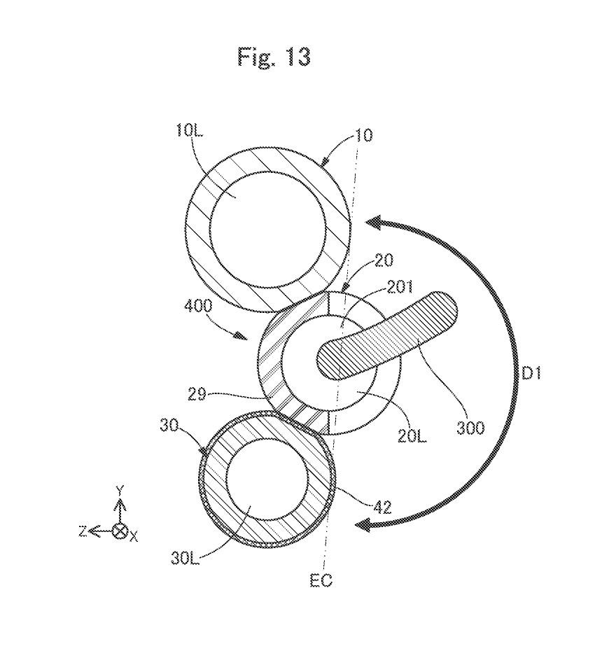

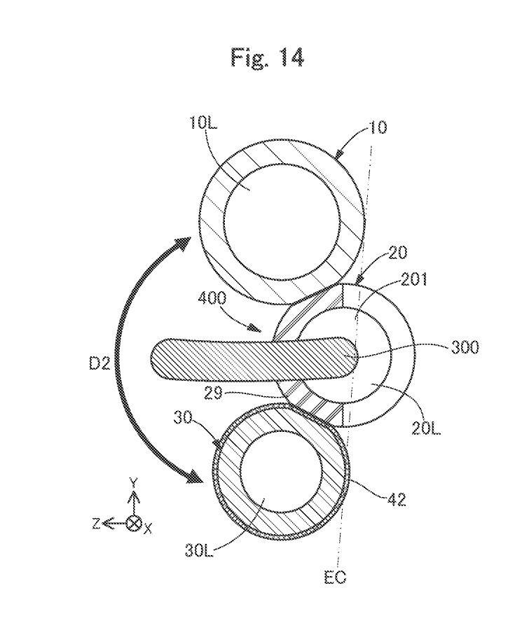

The length L400 of the gap 400 is the length between the gap distal end 401 and the gap proximal end 402. The length L400 of the gap 400 can be set arbitrarily, but is preferably longer than the length of the distal end opening 201 (the length between the distal end 201a and the proximal end 201b). The width Φ400 of the gap 400 is the length between the outer peripheral surface of the sensor tube 10 facing the gap 400 and the outer peripheral surface of the RX tube 30 facing the gap 400. The width Φ400 employed is the length of a portion where the sensor tube 10 and the RX tube 30 are farthest apart. The width Φ400 of the gap 400 can be set arbitrarily. For example, the width Φ400 of the gap 400 can be the same size as the outer diameter of the distal end of the OTW tube 20, or close to the outer diameter of the distal end of the OTW tube 20. In other words, the width Φ400 of the gap 400 can be the same as the outer diameter of a portion of the OTW tube 20, at which the distal end opening 201 is provided, or close to the outer diameter of a portion of the OTW tube 20, at which the distal end opening 201 is provided. Meanwhile, the width Φ400 of the gap 400 can be preferably the same size as the inner diameter of the distal end of the OTW tube 20, or close to the inner diameter of the distal end of the OTW tube 20. In other words, the width Φ400 of the gap 400 has preferably a size that is the same as or close to the inner diameter of the portion of the OTW tube 20, at which the distal end opening 201 is provided. The width Φ400 of the gap 400 may be smaller than the outer diameter of the therapeutic device 300 (see FIGS. 13 and 14) protruding from the OTW lumen 20L. The therapeutic device 300 often has an outer diameter that is close to the inner diameter of the OTW tube 20. So, for example, the width Φ400 of the gap 400 may be (inner diameter of the distal end of the OTW tube 20) * (1/2) < width Φ400 < (inner diameter of the distal end of the OTW tube 20) * (2/2). The sensor tube 10 and the RX tube 30 are formed of a resin material that can be elastically deformed relatively easily. Thus, for example, even if the width Φ400 of the gap 400 is smaller than the outer diameter of the therapeutic device 300 protruding from the OTW lumen 20L, when the surgeon pushes the therapeutic device 300 into the gap 400 from the proximal end side of the OTW lumen 20L, the sensor tube 10 and the RX tube 30 are elastically deformed so as to widen the gap 400. As a result, the surgeon can push the therapeutic device 300 forward into the gap 400.

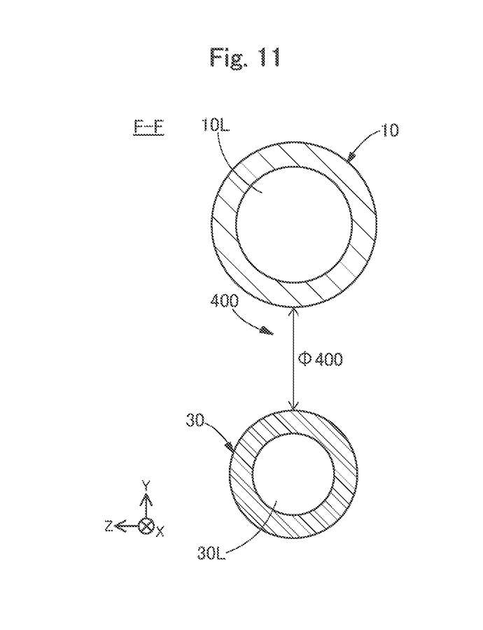

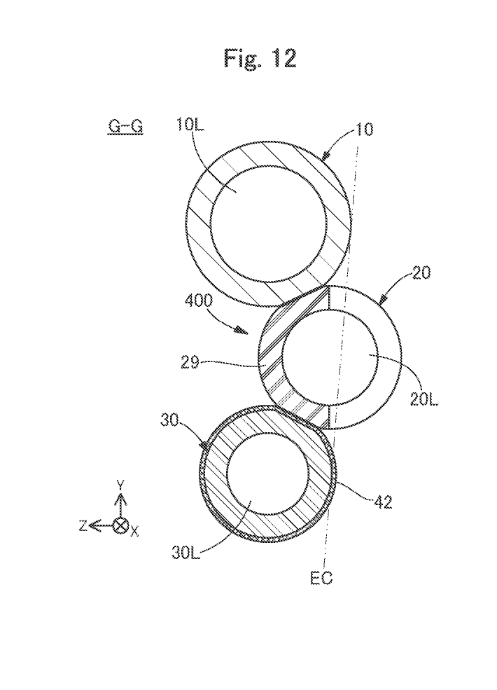

FIG. 11 is a transverse sectional view of the catheter 1 along line F-F in FIG. 10. Transverse section F-F shown in FIG. 11 shows a transverse section of the catheter 1 at the portion where the OTW tube 20 is absent. FIG. 12 is a transverse sectional view of the catheter 1 along line G-G in FIG. 10. Transverse section G-G shown in FIG. 12 shows a transverse section of the catheter 1 at the distal end portion of the OTW tube 20. In FIG. 12, of the two external common tangents of the sensor tube 10 and the RX tube 30, an external common tangent EC that is the one on the side closer to the OTW tube 20 is illustrated with a chain double-dashed line. The external common tangent EC corresponds to the “first external common tangent”.

As shown in FIG. 12, a portion of the distal end portion of the OTW tube 20 (second tube) enters the gap 400. The state in which “a portion of the distal end portion of the OTW tube 20 enters the gap 400” is described here. The transverse section of the catheter 1 at the position of a portion of the distal end portion of the OTW tube 20, i.e., where the gap 400 has a width Φ400, is referred to as the first transverse section. The transverse section of the catheter 1 at the position closer to the proximal end side than the gap 400 where the gap 400 is not formed, i.e., the transverse section of the catheter 1 at the position closer to the proximal end side than the gap 400, where the sensor tube 10 and the RX tube 30 are in contact with each other, is referred to as the second transverse section. “A portion of the distal end portion of the OTW tube 20 enters the gap 400” means that the OTW tube 20 in the first transverse section is located closer to the side of the sensor tube 10 and the RX tube 30 than the OTW tube 20 in the second transverse section, i.e., it enters the side of the sensor tube 10 and the RX tube 30. For example, it means that the position of the OTW tube 20 in the transverse section along line G-G in FIG. 12 is located closer to the side of the sensor tube 10 and the RX tube 30 than the position of the OTW tube 20 in the transverse section along line B-B in FIG. 4. In other words, in the longitudinal direction of the catheter 1, the OTW tube 20 is inclined toward the gap 400 from the position closer to the proximal end side than the gap 400, where the sensor tube 10 and the RX tube 30 are in contact, to the position where the gap 400 has the width Φ400. In the example of FIG. 12, of the thick-walled portion of the OTW tube 20, the outer portion on the sensor tube 10 (first tube) side and the outer portion of the thick-walled portion of the sensor tube 10 are in contact with each other, and deformed as illustrated in the figure. Of the thick-walled portion of the OTW tube 20, the outer portion on the RX tube 30 (third tube) side and the outer portion of the thick-walled portion of the RX tube 30 are in contact with each other, and deformed as illustrated in the figure. As illustrated in the figure, each of the tubes 10, 20, 30 can maintain the inner diameter of each of the lumens 10L, 20L, 30L because only the thick-walled portion is deformed.

Hereafter, of the distal end portion of the OTW tube 20, a portion located inside (i.e., on the side of the sensor tube 10 and the RX tube 30) of the external common tangent EC between the sensor tube 10 and the RX tube 30 is also referred to as a pinched portion 29. The length of the outer edge of the pinched portion 29 in transverse section G-G is 1/2 or more and 2/2 or less the length of the outer edge of the OTW tube 20. In order to allow the distal end portion to enter the gap 400, the distal end portion of the OTW tube 20 is inclined toward the distal end and the side of the sensor tube 10 and the RX tube 30 (FIG. 10: +Z axis direction).

In this embodiment, the distal end side tube 11 (first tube) of the sensor tube 10 is formed of a first material. The distal end side tube 21 (second tube) of the OTW tube 20 is formed of a third material. The RX tube 30 (third tube) is formed of a second material. The first material and the second material have different acoustic impedances from each other. The first material and the third material have different acoustic impedances from each other. The second material and the third material have different acoustic impedances from each other.

The gap 400 in the catheter 1 can be created as follows. First, an operator prepares the sensor tube 10, the OTW tube 20, the RX tube 30, and the core metal. The core metal has an outer diameter that is the same as or slightly smaller than the diameter of the OTW lumen 20L. The operator inserts the core metal into the OTW tube 20, with a portion on the distal end side of the core metal protruding outward from the distal end opening 201. The operator bundles and grasps the sensor tube 10, the RX tube 30, and the OTW tube 20 with the core metal inserted, and then passes the core metal protruding from the distal end opening 201 between the sensor tube 10 and the RX tube 30. In this state, the operator joins the three tubes 10, 20, 30.

FIG. 13 shows the therapeutic device 300 directed in the first direction D1. FIG. 14 shows the therapeutic device 300 directed in the second direction D2. FIG. 13 and FIG. 14 show transverse sectional views of the catheter 1 along line G-G in FIG. 10. Here, the first direction D1 means, with respect to the center of the OTW tube 20 (second tube), the direction that is the outside of the catheter 1 and toward the side opposite to the sensor tube 10 (first tube) and the RX tube 30 (third tube). In other words, the first direction D1 means the direction toward the side where the sensor tube 10 and the RX tube 30 are not placed, with respect to the external common tangent EC in FIG. 13. The second direction D2 means, with respect to the center of the OTW tube 20, the direction that is the outside of the catheter 1, and toward the side of the sensor tube 10 and the RX tube 30. In other words, the second direction D2 means the direction toward the side where the sensor tube 10 and the RX tube 30 are located, with respect to the external common tangent EC in FIG. 14.

As shown in FIG. 13, when the therapeutic device 300 is protruded from the distal end opening 201 (first distal end opening) (procedure a5), the surgeon directs the distal end portion of the therapeutic device 300 directly to the first direction D1 when the target site for treatment is in the first direction D1. Here, as described in FIG. 1, the distal end opening 201 is elliptical in shape with the distal end portion of the OTW tube 20 cut diagonally, and the distal end opening 201 faces the side of the first direction D1. This allows the surgeon to smoothly direct the distal end portion of the therapeutic device 300 to the first direction D1. Because the distal end opening 201 is elliptical in shape, when the distal end portion of the therapeutic device 300 is directed in the first direction D1, the distal end of the therapeutic device 300 protrudes from a position close to the proximal end 201b of the distal end opening 201 (i.e., the proximal end portion of the second marker 42). Thus, the surgeon can easily ascertain that the distal end portion of the therapeutic device 300 is facing in the first direction D1 based on the positional relationship between the distal end portion of the therapeutic device 300 and the second marker 42 in the image displayed on the console terminal.

As shown in FIG. 14, when the therapeutic device 300 is protruded from the distal end opening 201 (first distal end opening) (procedure a5), the surgeon directs the distal end portion of the therapeutic device 300 into the second direction D2 through the gap 400 when the target site for treatment is in the second direction D2. Here, as described in FIG. 12, a portion of the distal end portion of the OTW tube 20 enters the gap 400. Therefore, the distal end opening 201 has a depressed portion toward the side of the sensor tube 10 and the RX tube 30 (in other words, the second direction D2) at the position of the pinched portion 29. This allows the surgeon to push the distal end portion of the therapeutic device 300 forward along the depressed portion of the OTW tube at the position of the pinched portion 29. As a result, the surgeon can smoothly direct the distal end portion of the therapeutic device 300 into the second direction D2 through the gap 400. Because the distal end opening 201 is elliptical in shape, when the distal end portion of the therapeutic device 300 is directed to the second direction D2, the distal end portion of the therapeutic device 300 protrudes from a position close to the distal end 201a of the distal end opening 201 (i.e., the distal end portion of the second marker 42). Thus, the surgeon can easily ascertain that the distal end portion of the therapeutic device 300 is facing in the second direction D2 based on the positional relationship between the distal end portion of the therapeutic device 300 and the second marker 42 in the image displayed on the console terminal.

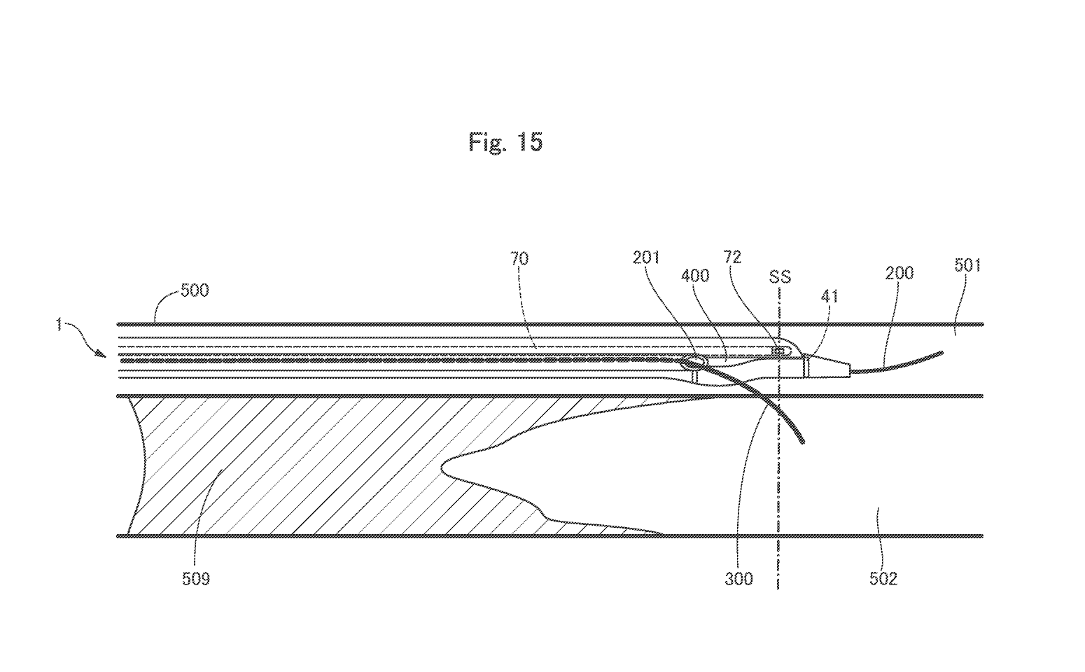

FIG. 15 shows the catheter 1 and combined devices in a first technique. The first technique is a technique to perform the approach from a false lumen 501 to a true lumen 502 after passage through CTO 509. The surgeon inserts the workhorse wire 200 into the catheter 1 as described in procedures a1 and a2 above. In procedure a3, the surgeon pushes the catheter 1 forward in the false lumen 501 until the distal end portion of the catheter 1 passes through the CTO 509. In procedure a4, the surgeon checks the position of the true lumen 502 using the probe 72 of the sensor 70. In procedure a5, the surgeon inserts the therapeutic device 300 into the catheter 1 and protrudes the distal end portion of the therapeutic device 300 from the distal end opening 201. In procedure a6, the surgeon uses the therapeutic device 300 to penetrate the intima of a blood vessel 500 and perform reentry from the false lumen 501 to the true lumen 502. In FIG. 15, for convenience of description, the orientation of the catheter 1 inserted into the blood vessel 500 in its own circumferential direction is the same as in FIG. 1. However, the orientation of the catheter 1 in the technique is not limited to the orientation shown in FIG. 15, but may be determined arbitrarily by the surgeon. The orientation of the catheter 1 in the technique may be rotated 90 degrees clockwise or counterclockwise around the circumference direction of the catheter 1 from the orientation shown in FIG. 15, for example.

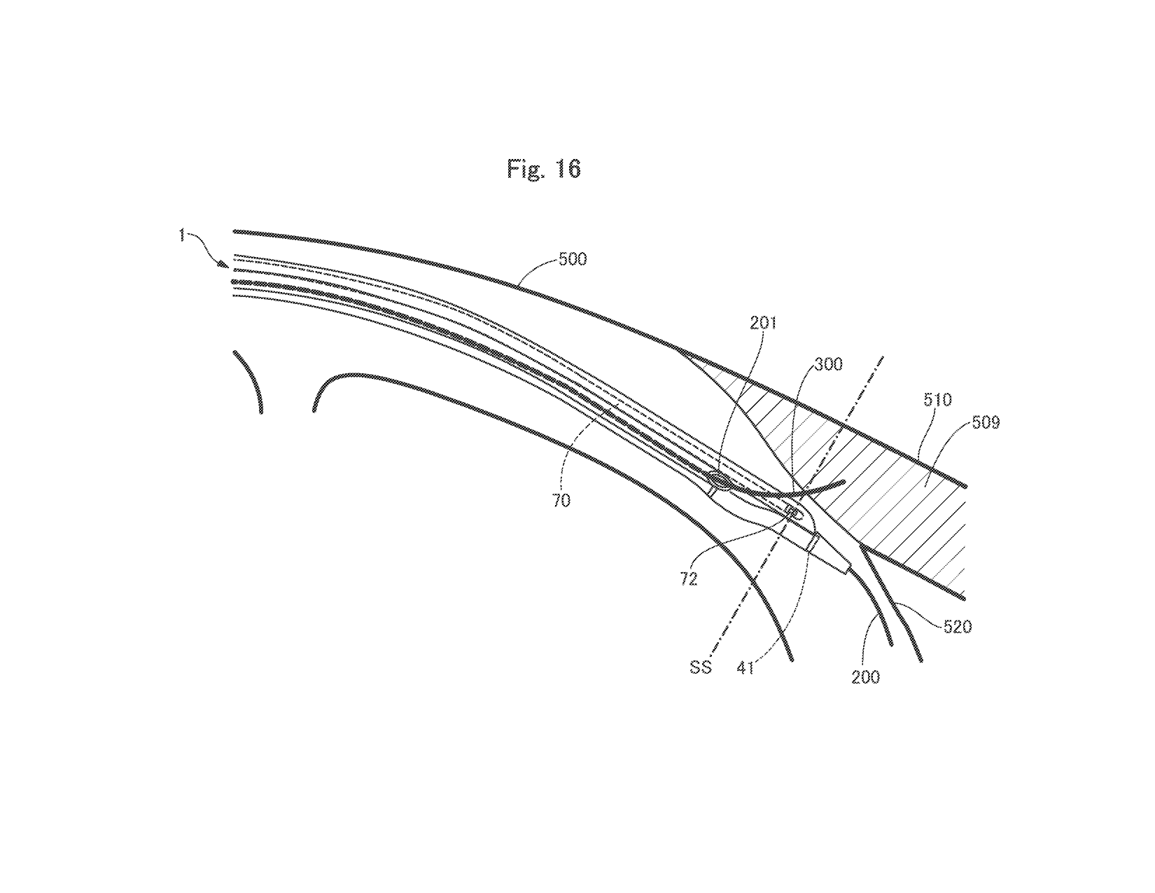

FIG. 16 illustrates the catheter 1 and combined devices in a second technique. The second technique is to penetrate CTO 509 existing at the entrance of a branching blood vessel that branches into a first 510 branch and a second 520 branch. The surgeon inserts the workhorse wire 200 into the catheter 1 as described in procedures a1 and a2 above. In procedure a3, the surgeon pushes the catheter 1 forward into the blood vessel 500 until the distal end portion of the catheter 1 is located on the near side of the CTO 509 formed in the first branch 510. In procedure a4, the surgeon checks the position of the first branch 510 and the CTO 509 using the probe 72 of the sensor 70. In procedure a5, the surgeon inserts the therapeutic device 300 into the catheter 1 and protrudes the distal end portion of the therapeutic device 300 from the distal end opening 201. In procedure a6, the surgeon uses the therapeutic device 300 to penetrate the CTO 509 and open the first branch 510. In FIG. 16, as in FIG. 15, the orientation of the catheter 1 inserted into the blood vessel 500 in its own circumferential direction is the same as in FIG. 1. However, the orientation of the catheter 1 in the technique is not limited to the orientation shown in FIG. 16, but may be determined arbitrarily by the surgeon.

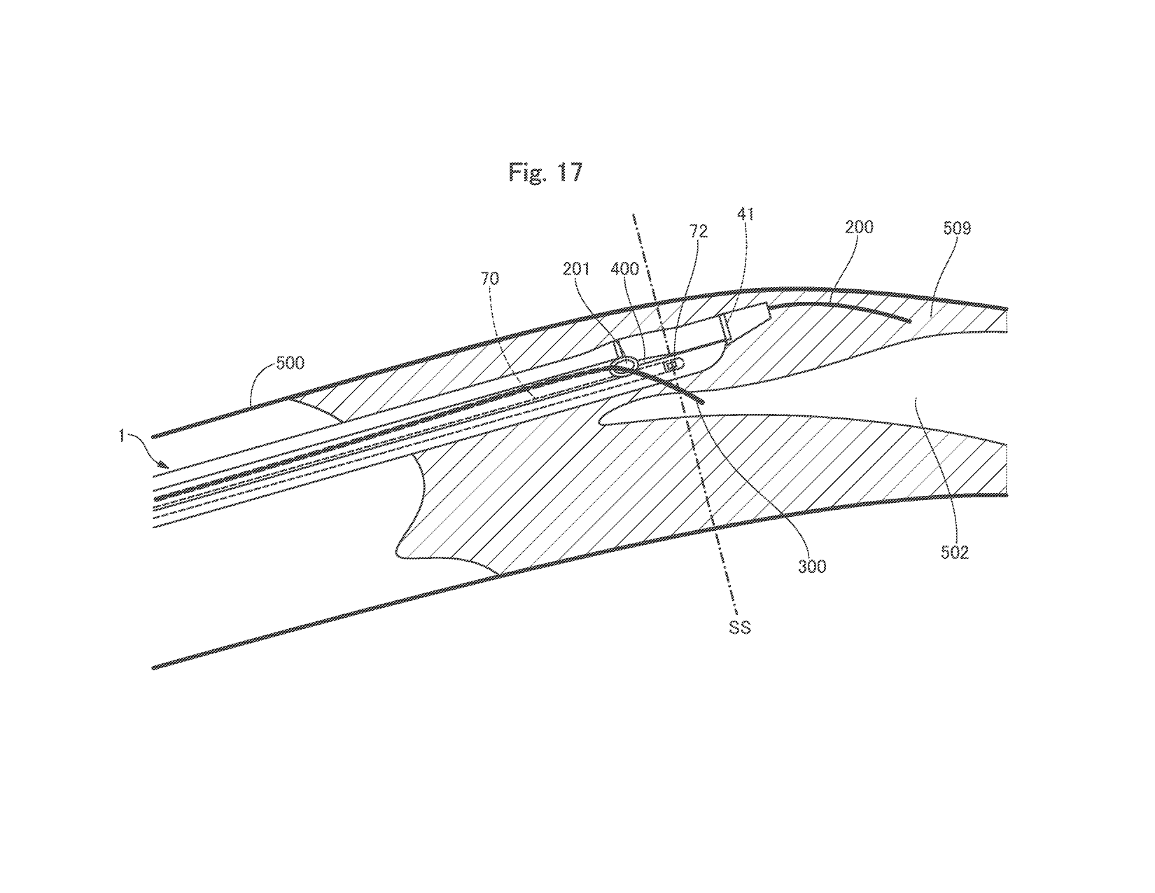

FIG. 17 shows the catheter 1 and combined devices in a third technique. The third technique is to penetrate the CTO 509 from the near side of the blood vessel 500. The surgeon inserts the workhorse wire 200 into the catheter 1 as described in procedures a1 and a2 above. In procedure a3, the surgeon pushes the catheter 1 forward to a position where the distal end portion of the catheter 1 is inside the CTO 509 that blocks the blood vessel 500, and the CTO 509 is easily penetrated (where the CTO 509 is depressed), given the shape of the CTO 509. The gap 400 is compressed by the external pressure applied from the CTO 509, and as shown in the figure, the width of the gap 400 changes to a smaller width, which facilitates pushing the distal end portion of the catheter 1 into the CTO 509. In procedure a4, the surgeon checks the position of the true lumen 502 in the depression of the CTO 509 using the probe 72 of the sensor 70. In procedure a5, the surgeon inserts the therapeutic device 300 into the catheter 1 and protrudes the distal end portion of the therapeutic device 300 from the distal end opening 201. In procedure a6, the surgeon uses the therapeutic device 300 to penetrate the CTO 509 for the distal end portion of the therapeutic device 300 to reach the true lumen 502. Also in FIG. 17, as in FIG. 15, the orientation of the catheter 1 inserted into the blood vessel 500 in its own circumferential direction is the same as in FIG. 1. However, the orientation of the catheter 1 in the technique is not limited to the orientation shown in FIG. 17, but may be determined arbitrarily by the surgeon.

In procedure a5 of FIGS. 15-17, the surgeon can direct the distal end portion of the therapeutic device 300 into the intended direction smoothly from directly the distal end opening 201 (FIG. 13) or through the gap 400 (FIG. 14), no matter where the target site for treatment (intima between the false lumen 501 and the true lumen 502) is located in the circumferential direction of the catheter 1. In FIGS. 15-17, the workhorse wire 200 can function as a landmark to ascertain the orientation of the catheter 1, since procedure a4 and onward is performed with the workhorse wire 200 inserted into the RX lumen 30L of the catheter 1. Furthermore, in FIGS. 15-17, the entry of the therapeutic device 300 into the biological tissue described in procedure a6 is performed at the position of the observation plane SS of the sensor 70, so that the safety of the technique can be improved.

As described above, according to the catheter 1 (medical device) of the first embodiment, at the position of the distal end opening 201 (first distal end opening), the gap 400 is formed between the sensor tube 10 (first tube) and the RX tube 30 (third tube). Therefore, when the therapeutic device 300 is inserted into the OTW lumen 20L (second lumen) and the distal end portion of the therapeutic device 300 is protruded from the distal end opening 201 (FIGS. 15-17), even if the target site for treatment is located on the side of the sensor tube 10 and the RX tube 30, the therapeutic device 300 can reach the target site through the gap 400 (FIG. 14). As a result, the medical device 1, with which the therapeutic device 300 in the lumen can be easily directed toward the target site for treatment, can be provided.

According to the catheter 1 (medical device) of the first embodiment, the distal end portion of the OTW tube 20 (second tube) is inclined toward the distal end and in the direction toward the gap 400. This allows the distal end portion of the therapeutic device 300 protruding from the distal end opening 201 (the first distal end opening) to pass more smoothly through the gap 400.

According to the catheter 1 (medical device) of the first embodiment, a portion of the distal end portion of the OTW tube 20 (second tube) enters the gap 400 of the sensor tube 10 and the RX tube 30 (FIG. 12). Therefore, the distal end portion of the OTW tube 20 is configured to be inclined toward the distal end and the side of the sensor tube 10 and the RX tube 30. As a result, this allows the distal end portion of the therapeutic device 300 protruding from the distal end opening 201 (the first distal end opening) to pass more smoothly through the gap 400 (FIG. 14).

Furthermore, according to the catheter 1 (medical device) of the first embodiment, the length of the outer edge of the pinched portion 29 is 1/2 or more and 2/2 or less the length of the outer edge of the distal end portion of the OTW tube 20 (second tube). This allows the distal end portion of the therapeutic device 300 protruding from the distal end opening 201 (the first distal end opening) to pass more smoothly through the gap 400.

Furthermore, according to the catheter 1 (medical device) of the first embodiment, the width Φ400 of the gap 400 is larger than the inner diameter of the distal end of the OTW tube 20 * (1/2) and smaller than the inner diameter of the distal end of the OTW tube 20 * (2/2). Therefore, even a therapeutic device 300 with an outer diameter that is close to the inner diameter of the OTW tube 20 (second tube) can be used.

Furthermore, according to the catheter 1 (medical device) of the first embodiment, when the distal end portion of the therapeutic device 300 is protruded from the distal end opening 201 (first distal end opening), even if the target site for treatment is located on the opposite side of the sensor tube 10 and the RX tube 30, the elliptical shape of the distal end opening 201 makes it easier to direct the distal end portion of the therapeutic device 300 toward the target site (FIG. 13). As a result, the usability of the catheter 1 can be further improved.

Furthermore, according to the catheter 1 (medical device) of the first embodiment, a portion of the outer peripheral surface of the sensor tube 10 (first tube) and a portion of the outer peripheral surface of the RX tube 30 (third tube) are in contact with each other on the distal end side (e.g. transverse section A-A) of the gap 400, allowing the distal end portion of the catheter 1 to be narrower in diameter. Further, the gap distal end 401 is located closer to the proximal end side than the distal end of the sensor tube 10, located closer to the distal end side than the distal end 201a of the distal end opening 201 (first distal end opening), and located closer to the proximal end side than the proximal end 201b of the distal end opening 201. This allows the length L400 of the gap 400 to be maintained sufficiently to facilitate passing the distal end portion of the therapeutic device 300 through the gap 400. As a result, the usability of the catheter 1 can be further improved.

Furthermore, according to the catheter 1 (medical device) of the first embodiment, the distal end of the RX lumen 30L (third lumen) is located closer to the distal end side than the distal end of the sensor lumen 10L (first lumen), allowing the distal end portion of the catheter 1 to be narrower in diameter. Since at the distal end of the RX lumen 30L, a distal end opening 301 (second distal end opening) for the RX lumen 30L to communicate with the outside is provided, the RX lumen 30L can be used as a lumen for a workhorse wire.

Furthermore, according to the catheter 1 (medical device) of the first embodiment, the first material forming the sensor tube 10 (first tube) and the second material forming the RX tube 30 (third tube) have different acoustic impedances from each other, and thus the sensor tube 10 and the RX tube 30 can be easily distinguished in the image acquired by the sensor. The first material forming the sensor tube 10 and the third material forming the OTW tube 20 (second tube) have different acoustic impedances from each other, and thus the sensor tube 10 and the OTW tube 20 can be easily distinguished in the image acquired by the sensor. Furthermore, the second material forming the RX tube 30 and the third material forming the OTW tube 20 have different acoustic impedances from each other, and thus the RX tube 30 and the OTW tube 20 can be easily distinguished in the image acquired by the sensor.

### Second Embodiment

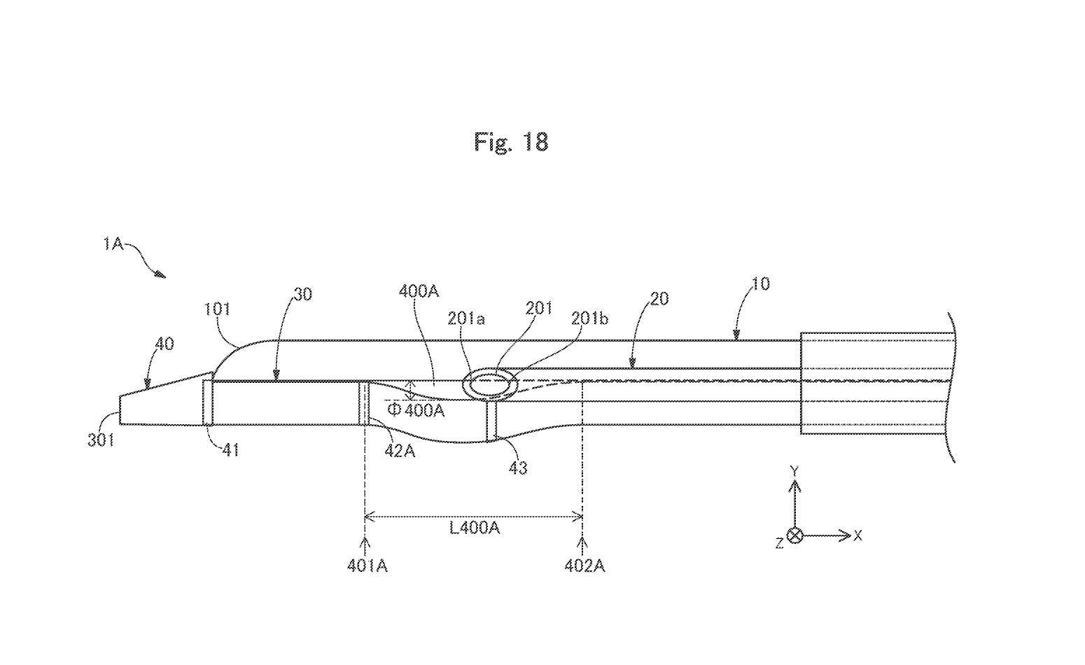

FIG. 18 is an enlarged view of a portion on the distal end side of a catheter 1A of the second embodiment. The catheter 1A of the second embodiment includes a gap 400A instead of the gap 400, a second marker 42A instead of the second marker 42, and a third marker 43 in the configuration described in the first embodiment.

As shown FIG. 18, a gap distal end 401 of the gap 400A is located away from the distal end of the sensor tube 10 toward the proximal end side and located closer to the distal end side than the distal end 201a of the distal end opening 201. The gap distal end 401A is located at the position where the second marker 42A is provided (specifically, in the center of the second marker 42A). Regarding the gap proximal end 402A of the gap 400A, the gap proximal end 402 is located closer to the proximal end side than the proximal end 201b of the distal end opening 201. In the second embodiment, the length L400A and width Φ400A of the gap 400A can be arbitrarily determined.

The second marker 42A and the third marker 43 are ring-shaped members having radiopacity. The second marker 42A is placed, so that the center of the second marker 42A and the gap distal end 401A are at the same position in the longitudinal direction of the catheter 1A. Specifically, the second marker 42A is a marker indicating the position of the gap distal end 401A. The third marker 43 is located so that the center of the third marker 43 and the center of the distal end opening 201 are at the same position in the longitudinal direction of the catheter 1A. Specifically, the third marker 43 is a marker indicating the position of the distal end opening 201. Similar to the first embodiment, it is more preferable for the second marker 42A and the third marker 43 to be made of metal.

Thus, the configuration of the catheter 1A can be changed in various ways, and the gap 400A may be provided together with a second marker 42A indicating the position of the gap 400A. The catheter 1A of the second embodiment can also be used in the first (FIG. 15), second (FIG. 16), and third (FIG. 17) techniques, respectively, as in the first embodiment. In the catheter 1A, the distal end opening 201 is located closer to the proximal end side of the catheter 1A compared to the configuration of the first embodiment, allowing the distal end portion of the catheter 1A to be narrower in diameter. Therefore, the catheter 1A is particularly suited for the third technique, in which the distal end portion is pushed forward to the interior of the CTO 509. Such catheter 1A of the second embodiment can also exert similar effects as in the above-described first embodiment. Furthermore, according to the catheter 1A of the second embodiment, the surgeon can easily grasp the position of the gap distal end 401A by checking images displayed on the console terminal.

### Third Embodiment

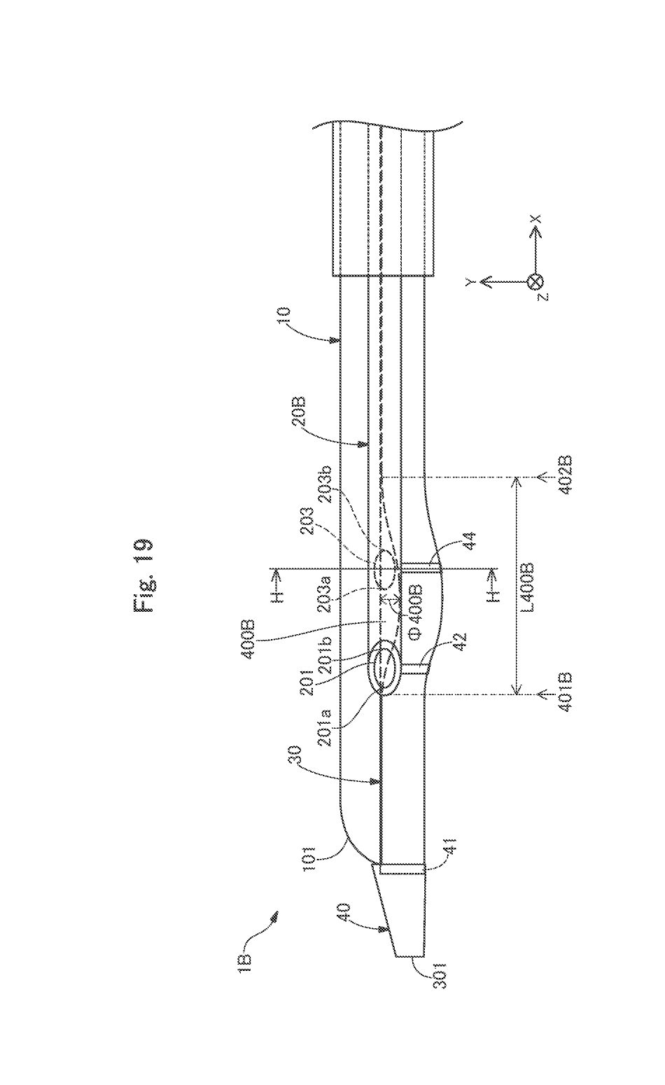

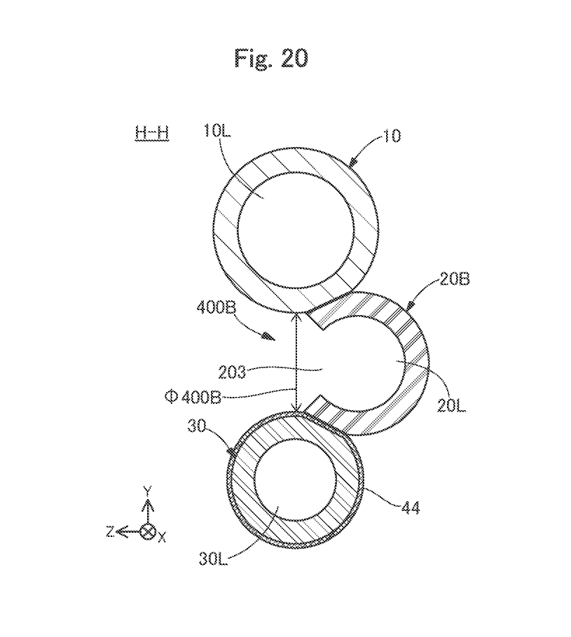

FIG. 19 is an enlarged view of a portion on the distal end side of a catheter 1B of the third embodiment. FIG. 20 is a transverse sectional view of the catheter 1B along line H-H in FIG. 19. The catheter 1A of the third embodiment includes an OTW tube 20B instead of the OTW tube 20, a gap 400B instead of the gap 400, and a fourth marker 44 in the configuration described in the first embodiment.

As shown in FIG. 19, the OTW tube 20B has a side opening 203 further in the configuration described in the first embodiment. The side opening 203 is, at a position away from the distal end opening 201 toward the proximal end side, a through hole that is provided in the side wall of the OTW tube 20B, for the OTW lumen 20L to communicate with the outside. The distal end 203a of the side opening 203 is located closer to the proximal end side than the proximal end 201b of the distal end opening 201. As shown in FIG. 20, the side opening 203 is provided at a position of the side wall of the OTW tube 20B, facing the gap 400B.

As shown in FIG. 19, the gap distal end 401B of the gap 400B is at the same position as the distal end 201a of the distal end opening 201. The gap proximal end 402B of the gap 400B is located closer to the proximal end side than the proximal end 203b of the side opening 203. In the third embodiment, the length L400B and the width Φ400B of the gap 400B can be arbitrarily determined. The fourth marker 44 is a ring-shaped member having radiopacity. The fourth marker 44 is placed so that the center of the fourth marker 44 and the center of the side opening 203 are at the same position in the longitudinal direction of the catheter 1B. Specifically, the fourth marker 44 is a marker indicating the position of the side opening 203. Similar to the first embodiment, it is more preferable for the fourth marker 44 to be made of metal.

Thus, the configuration of the catheter 1B can be changed in various ways, and the OTW tube 20B may further have a side opening 203 formed for protruding the therapeutic device 300 into the gap 400B. The catheter 1B of the third embodiment can also be used in the first (FIG. 15), second (FIG. 16), and third (FIG. 17) techniques, respectively, as in the first embodiment. Such catheter 1B of the third embodiment can also exert similar effects as in the above-described first embodiment.

Furthermore, according to the catheter 1B of the third embodiment, the use of the side opening 203 facing the gap 400B makes it even easier to pass the distal end portion of the therapeutic device 300 into the gap 400B (FIG. 20). The distal end opening 201 and the side opening 203 are located away from each other in the longitudinal direction of the catheter 1B. A second marker 42 is provided at the position of the distal end opening 201 and a fourth marker 44 is provided at the position of the side opening 203. Therefore, by checking the positional relationship of the therapeutic device 300, the second marker 42, and the fourth marker 44 in the image displayed on the console terminal, the surgeon can easily ascertain whether the therapeutic device 300 protrudes in the first direction D1 from the distal end opening 201 or whether the therapeutic device 300 protrudes in the second direction D2 from the side opening 203. Of course, the surgeon can also protrude the distal end portion of the therapeutic device 300 from the distal end opening 201 in the catheter 1B. As described above, according to the catheter 1B of the third embodiment, the OTW tube 20B (second tube) is further provided with a side opening 203 in the side wall at a position away from the distal end opening 201 (first distal end opening) toward the proximal end side, so that the therapeutic device 300 can also be protruded from the side opening. The side opening 203 is provided at a portion that is of the side wall of the OTW tube 20B and faces the gap 400B. This allows the therapeutic device 300 protruding from the side opening 203 to pass smoothly through the gap 400B. As a result, the usability of the catheter 1B can be further improved.

### Fourth embodiment

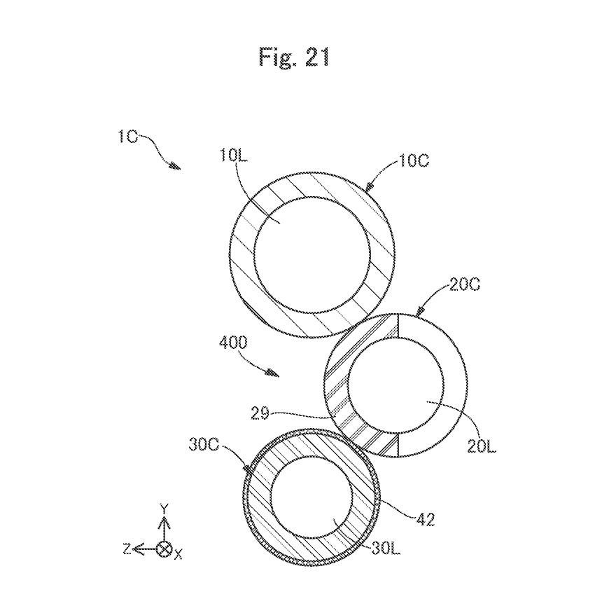

FIG. 21 is a transverse sectional view of the catheter 1C of the fourth embodiment. FIG. 21 corresponds to transverse section G-G in FIG. 10. The catheter 1C of the fourth embodiment includes a sensor tube 10C instead of the sensor tube 10, an OTW tube 20C instead of the OTW tube 20, and an RX tube 30C instead of the RX tube 30 in the configuration described in the first embodiment.

Regarding the OTW tube 20C, the distal end portion of the OTW tube 20C does not enter the gap 400. In other words, the position of the OTW tube 20C in the first transverse section is the same as the position of the OTW tube 20C in the second transverse section. The distal end portion of the OTW tube 20C is not inclined toward the side of the gap 400 and is parallel to the sensor tube 10C and the RX tube 30C. In other words, the sensor tube 10C, the OTW tube 20C, and the RX tube 30C are only in contact with each other's outer peripheral surface and have no deformation points in the thick-walled portion around the gap 400. In this embodiment, the distal end portion of the OTW tube 20C also has the pinched portion 29 provided and located inside the external common tangent EC between the sensor tube 10C and the RX tube 30C.

Thus, the configuration of the catheter 1C can be changed in various ways, and a configuration in which the distal end portion of the OTW tube 20C does not enter the gap 400 can be employed. The catheter 1C of the fourth embodiment can also be used in the first (FIG. 15), second (FIG. 16), and third (FIG. 17) techniques, respectively, as in the first embodiment. Such catheter 1C of the fourth embodiment can also exert similar effects as in the above-described first embodiment.

### Fifth embodiment

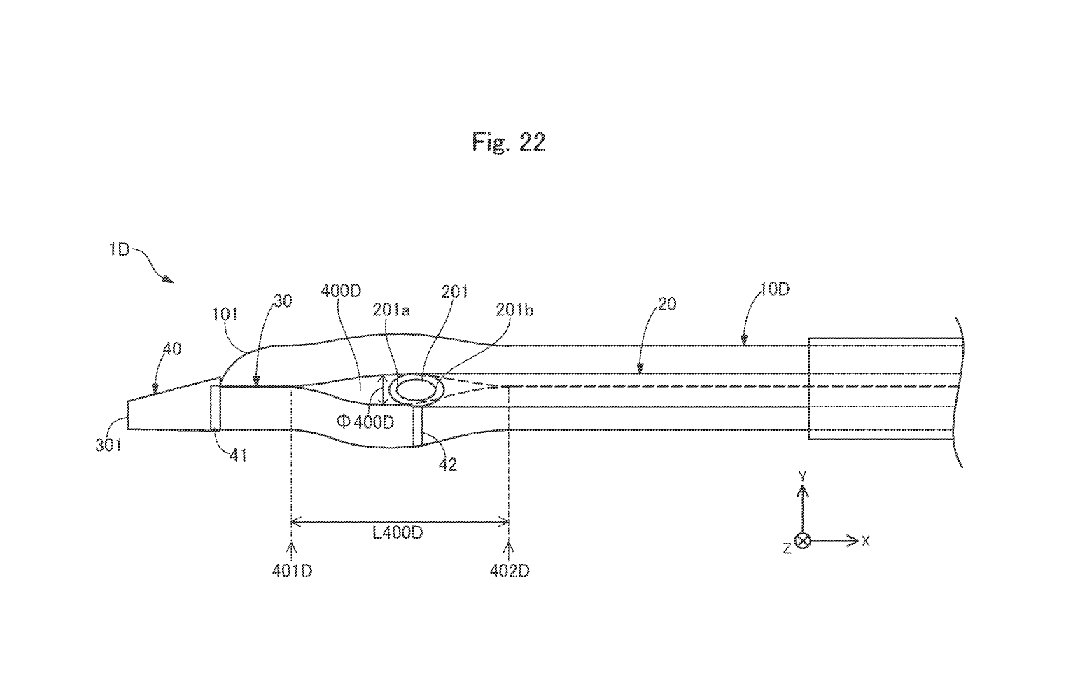

FIG. 22 is an enlarged view of a portion on the distal end side of a catheter 1D of the fifth embodiment. The catheter 1D of the fifth embodiment includes a sensor tube 10D instead of the sensor tube 10 and a gap 400D instead of the gap 400 in the configuration described in the first embodiment.

As shown in Fig, 22, a portion of the sensor tube 10D is curved in the direction away from the RX tube 30 (+Y axis direction). As in the first embodiment, a portion of the RX tube 30 is curved in the direction away from the sensor tube 10D (in the -Y axis direction). Thus, in the catheter 1D, the sensor tube 10D and the RX tube 30 are curved in the direction away from each other and thus separated from each other, so that the gap 400D is formed. The gap distal end 401D of the gap 400D and the gap proximal end 402D of the gap 400D are at the same position as in the first embodiment. In the fifth embodiment, the length L400D and the width Φ400D of the gap 400D can be arbitrarily determined.

Thus, the configuration of the catheter 1D can be changed in various ways. The gap 400D may be formed by curving both the sensor tube 10D and the RX tube 30. The RX tube 30 may be straight without curvature, and the gap 400D may also be formed by curving only the sensor tube 10D. The catheter 1D of the fifth embodiment can also be used in the first (FIG. 15), second (FIG. 16), and third (FIG. 17) techniques, respectively, as in the first embodiment. Such catheter 1D of the fifth embodiment can also exert similar effects as in the above-described first embodiment. In the catheter 1D of the fifth embodiment, the width Φ400D of the gap 400D can be larger than that in the configuration of the first embodiment. As a result, the catheter 1D makes it easier to pass the distal end portion of the therapeutic device 300 through the gap 400D.

### Modification examples of the embodiments

The disclosed embodiments are not limited to the above embodiments, and can be implemented in various aspects without departing from the gist thereof. For example, the following modification examples are also possible.

### Modification example 1

The above first to fifth embodiments show examples of the configurations of the catheters 1, and 1A to 1D. However, the configurations of the catheters 1, and 1A to 1D can be changed in various ways.

For example, the sensor tube 10 may be configured of a single tube without the distal end side tube 11 and the proximal end side tube 12. For example, the OTW tube 20 may be configured of a single tube without the distal end side tube 21 and the proximal end side tube 22. For example, the sensor tube 10, the OTW tube 20, and the RX tube 30 may be molded as one piece. Even in the case of integral molding, by providing a through hole that penetrates the shaft in the Z-axis direction in the wall thickness portion of the shaft between the sensor lumen 10L and the RX lumen 30L at the position of the distal end opening 201, the through hole can function as a gap to achieve the similar effects as in the first embodiment.

For example, the catheter 1 may be further provided with a coating portion to prevent the expansion of the gaps 400, 400A, 400B, 400D with use. The coating portion can comprise at least one of the distal end side coating portion and the proximal end side coating portion. A distal end side coating portion is a layer that is located closer to the distal end side than the gap distal end, and covers the outer peripheral surface of the sensor tube 10 and the RX tube 30. The proximal end side coating portion is a layer that is located closer to the proximal end side than the gap proximal end, and covers the outer peripheral surface of the sensor tube 10, the OTW tube 20, and the RX tube 30. The coating portion can be formed with the use of a coating agent or an adhesive. With the coating portion formed with a coating agent, the expansion of the gap 400 can be prevented while maintaining flexibility.

For example, the distal end opening 201 (first distal end opening) of the OTW lumen 20L may be circular with the distal end portion of the OTW tube 20 cut vertically. Similarly, the proximal end opening 302 of the RX lumen 30L may be circular with the proximal end portion of the RX tube 30 cut vertically. For example, the distal end opening 201 of the OTW lumen 20L may be located closer to the distal end side than the distal end opening 301 of the RX lumen 30L, and the distal end opening 301 of the RX lumen 30L may be located closer to the proximal end side than the acoustic window AW.

For example, at least two of the sensor tube 10, the OTW tube 20, and the RX tube 30 may be formed of materials having the same acoustic impedance as each other. Even if the three tubes 10, 20, 30 are formed of materials having the same acoustic impedance, the thickness of each of tubes 10, 20, 30 can be made different from each other to identify each of tubes 10, 20, 30 in the image acquired by the sensor 70.

For example, the sensor 70 is built into the sensor lumen 10L of the sensor tube 10 and is not removable from the catheter 1. However, the sensor 70 may be configured to be removable from the catheter 1. Specifically, the catheter 1 does not have to include sensor 70 as a component. For example, the outer peripheral surface of the first outer tube 50, the heat shrink tube 90, and the second outer tube 80, or the outer peripheral surface of the catheter 1 including them, may be coated with a hydrophilic or hydrophobic resin.

For example, at least one of the distal tip 40, the first marker 41, the second marker 42, the third marker 43, and the fourth marker 44 may be omitted. The placement and shapes of these can be changed as desired. For example, the first marker 41 may not overlap the distal tip 40 and may be placed at a position adjacent to or away from the proximal end of the distal tip 40. For example, at least one of the first through fourth markers 41, 42, 43, 44 may be placed on a tube different from the RX tube 30 (the sensor tube 10 or the OTW tube 20). For example, the distal tip 40 may have a constant outer diameter from the distal end to the proximal end, and the shape of the transverse section may be non-circularly symmetrical. For example, at least one of the first through fourth markers 41, 42, 43, and 44 may have a shape different from a ring shape (e.g., a circular ring cut at an arbitrary angle, a linear shape, or a coil formed of a wire wound spirally around).

For example, at least one of the first outer tube 50, the second outer tube 80, and the heat shrink tube 90 may be omitted. For example, the shapes of the branch connector 60, the first reinforcement member 61 to the third reinforcement member 63, the outer tubular member 64, the inner tubular member 67, the connector 65, and the connector 25 described above are only examples and may be changed as desired. For example, at least a portion of the branch connector 60, the first reinforcement member 61, the second reinforcement member 62, and the outer tubular member 64 and the inner tubular member 67 may be configured as a single member or may be omitted. For example, the third reinforcement member 63 and the connector 25 may be configured as a single member. For example, the outer tubular member 64 may be formed of a transparent material, and the inner tubular member 67 may include a mechanism (e.g., scales or stoppers provided at predetermined lengths in the longitudinal direction, scales or stoppers provided at predetermined angles in the circumferential direction) to assist in adjusting at least one of the front-back positions of the sensor 70 and the direction of the sensor 70 in the circumferential direction.).

### Modification example 2

The configurations of the catheters 1, and 1A to 1D of the first to fifth embodiments, and the configurations of the catheters 1, and 1A to 1D of the above-described modification example 1 may be combined appropriately. For example, in any of the catheters 1A, 1B described in the second embodiment or the third embodiment, the catheter may include the OTW tube 20C described in the fourth embodiment or the sensor tube 10D described in the fifth embodiment.

The present aspects are described above on the basis of the embodiments and the modification examples. However, the embodiments of the aforementioned aspects are provided to facilitate the understanding of the present aspects, and do not limit the present aspects. The present aspects may be modified or improved without departing from the spirit thereof and claims, and the present aspects include their equivalents. Further, the technical features thereof, if not indicated as essential in the present specification, may be appropriately deleted.

The disclosed embodiments are made in order to solve at least some of the above problems, and can be implemented according to the following aspects.

(1) According to one aspect, a medical device is provided. The medical device includes a first tube having a first lumen, a second tube placed alongside the first tube and having a second lumen, and a third tube placed alongside the first and second tubes and having a third lumen, wherein the distal end of the second tube is placed closer to the proximal end side than the distal end of the first tube and closer to the proximal end side than the distal end of the third tube, a first distal end opening for the second lumen to communicate with the outside is formed at the distal end of the second tube, and at the position of the first distal end opening, the first tube and the third tube are separated from each other to form a gap between the first tube and the third tube.

According to this configuration, a gap is formed between the first tube and the third tube at the position of the first distal end opening. Therefore, when a therapeutic device is inserted into the second lumen and the distal end portion of the therapeutic device is protruded from the first distal end opening, even if the target site for treatment is located on the side of the first and third tubes, the therapeutic device can reach the target site by passing through the gap between the first and third tubes. As a result, the medical device, with which the therapeutic device in the lumen can be easily directed toward the target site for treatment, can be provided.

(2) In the medical device of the above aspect, the distal end portion of the second tube may be inclined toward the distal end and in the direction toward the gap.

According to this configuration, the distal end portion of the second tube is inclined toward the distal end and in the direction toward the gap. This allows the distal end portion of the therapeutic device protruding from the first distal end opening to pass more smoothly through the gap between the first and third tubes.

(3) In the medical device of the above aspect, a portion of the distal end portion of the second tube may enter the gap.

According to this configuration, a portion of the distal end portion of the second tube enters the gap between the first and third tubes, so that the distal end portion of the second tube is inclined toward the distal end and the side of the first and third tubes. This allows the distal end portion of the therapeutic device protruding from the first distal end opening to pass more smoothly through the gap between the first and third tubes.

(4) In the medical device of the above aspect, in the transverse section of the medical device at the position where the gap is formed, and when, of external common tangents between the first tube and the third tube, an external common tangent closer to the second tube is designated as a first external common tangent, the length of the outer edge of a pinched portion that is of the distal end portion of the second tube, and is located inside the first common tangent, may be 1/2 or more and 2/2 or less the length of the outer edge of the distal end portion of the second tube.

According to this configuration, the length of the outer edge of the pinched portion is 1/2 or more and 2/2 or less the length of the outer edge of the distal end portion of the second tube. Accordingly, the distal end portion of the therapeutic device protruding from the first distal end opening can pass more smoothly through the gap between the first and the third tubes.

(5) In the medical device of the above aspect, the width of the gap may be larger than the inner diameter of the distal end of the second tube * (1/2) and smaller than the inner diameter of the distal end of the second tube * (2/2).

According to this configuration, the width of the gap is larger than the inner diameter of the distal end of the second tube * (1/2) and smaller than the inner diameter of the distal end of the second tube * (2/2). Thus, even a therapeutic device with an outer diameter close to the inner diameter of the second tube can be used.

(6) In the medical device of the above aspect, the first distal end opening may have an elliptical shape formed by cutting diagonally the distal end portion of the second tube.

According to this configuration, when the distal end portion of a therapeutic device is protruded from the first distal end opening, even if the target site for treatment is located on the opposite side of the first and third tubes, the elliptical shape of the first distal end opening makes it easier to direct the distal end portion of the therapeutic device toward the target site. As a result, the usability of the medical device can be further improved.

(7) In the medical device of the above aspect, a portion of the outer peripheral surface of the first tube and a portion of the outer peripheral surface of the third tube are in contact with each other at a position closer to the distal end side than the gap, and the distal end of the gap may be located closer to the proximal end side than the distal end of the first tube, and, located closer to the distal end side than the distal end of the first distal end opening, and the proximal end of the gap may be located closer to the proximal end side than the proximal end of the first distal end opening.

According to this configuration, a portion of the outer peripheral surface of the first tube and a portion of the outer peripheral surface of the third tube are in contact with each other at a position closer to the distal end side than the gap, allowing the distal end portion of the medical device to be narrower in diameter. Further, the distal end of the gap is located closer to the proximal end side than the distal end of the first tube, located closer to the distal end side than the distal end of the first distal end opening, and the proximal end of the gap is located closer to the proximal end side than the proximal end of the first distal end opening, making it easier to pass the distal end portion of the therapeutic device through the gap. As a result, the usability of the medical device can be further improved.

(8) In the medical device of the above aspect, the distal end of the third lumen is located closer to the distal end side than the distal end of the first lumen, and a second distal end opening for the third lumen to communicate with the outside may be provided at the distal end of the third lumen.

According to this configuration, the distal end of the third lumen is located closer to the distal end side than the distal end of the first lumen, allowing the distal end portion of the medical device to be narrower in diameter. A second distal end opening for the third lumen to communicate with the outside is provided at the distal end of the third lumen, allowing the third lumen to be used as a lumen for a workhorse wire.

(9) In the medical device of the above aspect, the second tube may further be provided with a side opening for the second lumen to communicate with the outside at a side wall located away from the first distal end opening toward the proximal end side.

According to this configuration, the second tube is further provided with a side opening in the side wall located away from the first distal end opening toward the proximal end side, so that the therapeutic device can also protrude from the side opening. As a result, the usability of the medical device can be further improved.

(10) In the medical device of the above aspect, the side opening may be provided at a portion that is of the side wall of the second tube, and faces the gap.

According to this configuration, the side opening is provided at the portion that is of the side wall of the second tube, and faces the gap. This allows the therapeutic device protruding from the side opening to pass smoothly through the gap.

(11) In the medical device of the above aspect, the first tube may be formed of a first material, the third tube may be formed of a second material, and the first material and the second material may have different acoustic impedances.

According to this configuration, the first material and the second material have different acoustic impedances from each other, so the first tube and the third tube can be easily distinguished in the image acquired by the sensor.

(12) In the medical device of the above aspect, the second tube may be formed of a third material, and the first material and the third material may have different acoustic impedances.

According to this configuration, the first material and the third material have different acoustic impedances from each other, so the first tube and second tube can be easily distinguished in the image acquired by the sensor.

(13) In the medical device of the above aspect, the second material and the third material may have different acoustic impedances.

According to this configuration, the second material and the third material have different acoustic impedances from each other, so the third tube and the second tube can be easily distinguished in the image acquired by the sensor.

The disclosed embodiments can be realized in various aspects, for example, in the form of medical devices, medical tubes, catheters, and methods of manufacturing these devices.

### REFERENCE SIGNS LIST

1, 1A-1D...Catheter (medical device)
10, 10D...Sensor tube (first tube)
11...Distal end side tube
12...Proximal end side tube
20, 20B, 20C...OTW tube (second tube)
21...Distal end side tube
22...Proximal end side tube
25...Connector
29...Pinched portion
30...RX tube (third tube)
40...Distal tip
41...First marker
42, 42A...Second marker
43...Third marker
44...Fourth marker
50...First outer tube
60...Branch connector
61...First reinforcement member
62...Second reinforcement member
63...Third reinforcement member
64...Outer tubular member
65...Connector
66...Fluid supply part
67...Inner tubular member
70...Sensor
71...Main body part
72...Probe
75...Connection terminal
80...Second outer tube
90...Heat shrink tube
101...Distal end opening
102...Proximal end opening
200...Work horse wire
201...Distal end opening
202...Proximal end opening
203...Side opening
300...Therapeutic device
301...Distal end opening
302...Proximal end opening
400, 400A, 400B, 400D... Gap
401, 401A, 401B, 401D... Gap distal end
402, 402A, 402B, 402D... Gap proximal end

What is claimed is:

1. A medical device, comprising:
a first tube including a first lumen;
a second tube placed alongside the first tube and including a second lumen; and
a third tube placed alongside the first tube and the second tube and including a third lumen, wherein:
a distal end of the second tube is placed closer to a proximal end side than a distal end of the first tube and closer to a proximal end side than a distal end of the third tube;
a first distal end opening for the second lumen to communicate with the outside of the medical device is formed at the distal end of the second tube; and
at the first distal end opening, the first tube and the third tube are separated from each other to form a gap between the first tube and the third tube.

2. The medical device according to claim 1, wherein a distal end portion of the second tube is inclined toward a distal end of the medical device and in a direction toward the gap.

3. The medical device according to claim 1 wherein a portion of the distal end portion of the second tube enters the gap.

4. The medical device according to claim 3, wherein:
in a transverse section of the medical device at a position where the gap is formed, the first tube and the third tube define two external common tangents that each extend between respective outer surfaces of the first tube and the third tube, and an external common tangent of the two external common tangents that is located closest to the second tube is designated as a first external common tangent; and
the distal end portion of the second tube has a pinched portion that is located on the same side of the first external common tangent as the first tube and the third tube, and the pinched portion has an outer edge with a length that is from one half to the entire length of an outer edge of the distal end portion of the second tube.

5. The medical device according to claim 1, wherein a width of the gap is larger than one half of an inner diameter of the distal end of the second tube and smaller than the entire inner diameter of the distal end of the second tube.

6. The medical device according to claim 1, wherein the first distal end opening has an elliptical shape formed by cutting diagonally the distal end portion of the second tube.

7. The medical device according to claim 6, wherein:
a portion of an outer peripheral surface of the first tube and a portion of an outer peripheral surface of the third tube are in contact with each other at a position closer to the distal end side of the medical device than the gap; and
a distal end of the gap is located closer to the proximal end side than the distal end of the first tube and closer to a distal end side than a distal end of the first distal end opening; and
the proximal end of the gap is located closer to the proximal end side than a proximal end of the first distal end opening.

8. The medical device according to claim 1, wherein:
a distal end of the third lumen is located closer to a distal end side than a distal end of the first lumen; and
a second distal end opening for the third lumen to communicate with the outside of the medical device is provided at the distal end of the third lumen.

9. The medical device according to claim 1 wherein the second tube is further provided with a side opening for the second lumen to communicate with the outside at a side wall located away from the first distal end opening toward the proximal end side of the medical device.

10. The medical device according to claim 9, wherein the side opening is provided at a portion of a side wall of the second tube, and the side opening faces the gap.

11. The medical device according to claim 1 wherein:
the first tube is formed of a first material;
the third tube is formed of a second material; and
the first material and the second material have different acoustic impedances.

12. The medical device according to claim 11, wherein:
the second tube is formed of a third material; and
the first material and the third material have different acoustic impedances.

13. The medical device according to claim 12, wherein the second material and the third material have different acoustic impedances.

14. The medical device according to claim 2 wherein a portion of the distal end portion of the second tube enters the gap.

15. The medical device according to claim 14, wherein:
in a transverse section of the medical device at a position where the gap is formed, the first tube and the third tube define two external common tangents that extend between respective outer surfaces of the first tube and the third tube, and an external common tangent of the two external common tangents that is located closest to the second tube is designated as a first external common tangent; and
the distal end portion of the second tube has a pinched portion that is located on the same side of the first external common tangent as the first tube and the third tube, and the pinched portion has an outer edge with a length that is from one half to the entire length of an outer edge of the distal end portion of the second tube.

16. The medical device according to claim 2, wherein a width of the gap is larger than one half of an inner diameter of the distal end of the second tube and smaller than the entire inner diameter of the distal end of the second tube.

17. The medical device according to claim 2, wherein the first distal end opening has an elliptical shape formed by cutting diagonally the distal end portion of the second tube.

18. The medical device according to claim 17, wherein:
a portion of an outer peripheral surface of the first tube and a portion of an outer peripheral surface of the third tube are in contact with each other at a position closer to the distal end side of the medical device than the gap; and
a distal end of the gap is located closer to the proximal end side than the distal end of the first tube and closer to a distal end side than a distal end of the first distal end opening; and
the proximal end of the gap is located closer to the proximal end side than a proximal end of the first distal end opening.

19. The medical device according to claim 2, wherein:
a distal end of the third lumen is located closer to a distal end side than a distal end of the first lumen; and
a second distal end opening for the third lumen to communicate with the outside of the medical device is provided at the distal end of the third lumen.

20. The medical device according to claim 2 wherein the second tube is further provided with a side opening for the second lumen to communicate with the outside at a side wall located away from the first distal end opening toward the proximal end side of the medical device.
